(19) United States
(12) Patent Application Publication
(10) Pub. No.: US 2006/0117409 A1
(43) Pub. Date: Jun. 1, 2006

(54) Modified ribulose 1,5-bisphosphate carboxylase/oxygenase for improvement and optimization of plant phenotypes

(75) Inventors: Willem P.C. Stemmer, Los Gatos, CA (US); Venkitswaran Subramanian, San Diego, CA (US); Genhai Zhu, Sunnyvale, CA (US); Lu Liu, Redwood City, CA (US); Sergey A. Selifonov, Los Altos, CA (US)

Correspondence Address: MCANDREWS HELD & MALLOY, LTD, 500 WEST MADISON STREET, SUITE 3400, CHICAGO, IL, 60661, US

(73) Assignee: Maxygen, Inc., Redwood City, CA (US)

(21) Appl. No.: 11/291,311

(22) Filed: Dec. 1, 2005

Related U.S. Application Data
(63) Continuation of application No. 09/437,726, filed on Nov. 9, 1999, now abandoned.
(60) Provisional application No. 60/153,093, filed on Sep. 9, 1999.
(60) Provisional application No. 60/107,756, filed on Nov. 10, 1998.

(51) Int. Cl. A01H 1/00; C40B 40/08; C12Q 1/68

(52) U.S. Cl. 800/278; 435/6

(57) ABSTRACT

The invention relates to methods and compositions for generating, modifying, adapting, and optimizing polynucleotide sequences that encode proteins having Rubisco biosynthetic enzyme activities which are useful for introduction into plant species, agronomically-important microorganisms, and other hosts, and related aspects.

### CROSS REFERENCE TO RELATED APPLICATIONS

This application is a non-provisional filing of and claims priority to “MODIFIED RIBULOSE 1,5-BISPHOSPHATE CARBOXYLASE/OXYGENASE FOR IMPROVEMENT AND OPTIMIZATION OF PLANT PHENOTYPES” by Stemmer et al., U.S. Ser. No. 60/153,093, filed Sep. 9, 1999 and to “MODIFIED RIBULOSE 1,5-BISPHOSPHATE CARBOXYLASE/OXYGENASE FOR IMPROVEMENT AND OPTIMIZATION OF PLANT PHENOTYPES” by Stemmer et al., U.S. Ser. No. 60/107,756, filed Nov. 10, 1998.

### FIELD OF THE INVENTION

The invention relates to methods and compositions for generating, modifying, adapting, and optimizing polynucleotide sequences that encode proteins having Rubisco biosynthetic enzyme activities which are useful for introduction into plant species, agronomically-important microorganisms, other hosts and related aspects.

### BACKGROUND

### Genetic Engineering of Plants

Genetic engineering of agricultural organisms dates back thousands of years to the dawn of agriculture. The hand of man has selected the agricultural organisms having the phenotypic traits that were deemed desirable, which desired phenotypic traits have often been taste, high yield, caloric value, ease of propagation, resistance to pests and disease, and appearance. Classical breeding methods to select for germplasm encoding desirable agricultural traits had been a standard practice of the world's farmers long before Gregor Mendel and others identified the basic rules of segregation and selection. For the most part, the fundamental process underlying the generation and selection of desired traits was the natural mutation frequency and recombination rates of the organisms, which are quite slow compared to the human lifespan and make it difficult to use conventional methods of breeding to rapidly obtain or optimize desired traits in an organism.

The relatively recent advent of non-classical, or “recombinant” genetic engineering techniques has provided a new means to expedite the generation of agricultural organisms having desired traits that provide an economic, ecological, nutritional, or aesthetic benefit. To date, most recombinant approaches have involved transferring a novel or modified gene into the germline of an organism to effect its expression or to inhibit the expression of the endogenous homologue gene in the organism's native genome. However, the currently used recombinant techniques are generally unsuited for substantially increasing the rate at which a novel or improved phenotypic trait can be evolved. Essentially all recombinant genes in use today for agriculture are obtained from the germplasm of existing plant and microbial specimens, which have naturally evolved coordinately with constraints related to other aspects of the organism's evolution and typically are not specifically optimized for the desired phenotype(s). The sequence diversity available is limited by the natural genetic variability within the existing specimen gene pool, although crude mutagenic approaches have been used to add to the natural variability in the gene pool.

Unfortunately, the induction of mutations to generate diversity often requires chemical mutagenesis, radiation mutagenesis, tissue culture techniques, or mutagenic genetic stocks. These methods provide means for increasing genetic variability in the desired genes, but frequently produce deleterious mutations in many other genes. These other traits may be removed, in some instances, by further genetic manipulation (e.g., backcrossing), but such work is generally both expensive and time consuming. For example, in the flower business, the properties of stem strength and length, disease resistance and maintaining quality are important, but often initially compromised in the mutagenesis process.

### Ribulose 1,5-Bisphosphate Carboxylase/Oxygenase

Carbon fixation, or the conversion of CO₂ to reduced forms amenable to cellular biochemistry, occurs by several metabolic pathways in diverse organisms. The most familiar of these is the Calvin Cycle (or “Calvin-Benson” cycle), which is present in cyanobacteria and their plastid derivatives (i.e., chloroplasts), as well as in proteobacteria. The Calvin cycle utilizes, e.g., the enzyme rubisco (ribulose-1,5-bisphosphate carboxylase/oxygenase). Rubisco exists in at least two forms: form I rubisco is found in proteobacteria, cyanobacteria, and plastids, e.g., as an octo-dimer composed of eight large subunits, and eight small subunits; form II rubisco is a dimeric form of the enzyme, e.g., as found in proteobacteria. Form I rubisco is encoded by two genes (rbcL and rbcS,) while form II rubisco has clear similarities to the large subunit of form I rubisco, and is encoded by a single gene, also called rbcL. The evolutionary origin of the small subunit of form I rubisco remains uncertain; it is less highly conserved than the large subunit, and may have cryptic homology to a portion of the form II protein. See, e.g., http://www.blc.arizona.edu/courses/181 gh/rick/photosynthesis/Calvin.html, or Raven et al. (1981) The Biology of Plants, 3^{rd} Edition Worth Publishers, Inc. NY, N.Y. for a discussion of the Calvin Cycle. Because of the abundance of Rubisco in Chloroplasts (at about 15% of total protein), it is often indicated to be the most abundant protein on each (Raven et al., id.).

All photosynthetic organisms catalyze the fixation of atmospheric CO₂ by the bifunctional enzyme ribulose 1,5-bisphosphate carboxylase/oxygenase (“Rubisco”; EC 4.1.1.39). Significant variations in kinetic properties of this enzyme are found among various phylogenetic groups. Because of the abundance and fundamental importance of Rubisco, the enzyme has been extensively studied. Well over 1,000 different Rubisco homologues are available in the public literature (e.g., over 1,000 different Rubisco homologues are listen in GenBank alone), and the crystal structure of Rubisco has been solved for several variants of the protein.

Rubisco contains two competing enzymatic activities: an oxygenase and a carboxylase activity. The oxygenation reaction catalyzed by Rubisco is a “wasteful” process since it competes with and significantly reduces the net amount of carbon fixed. The Rubisco enzyme species encoded in various photosynthetic organisms have been selected by natural evolution to provide higher plants with a Rubisco enzyme that is substantially more efficient at carboxylation in the presence of atmospheric oxygen. Nonetheless, there remains a substantial range for improvement of the Rubisco enzyme to improve the carboxylation specificity.

As noted, the advent of recombinant DNA technology has provided agriculturists with additional means of modifying plant genomes. While certainly practical in some areas, to date genetic engineering methods have had limited success in transferring or modifying important biosynthetic or other pathways, including the Rubisco enzyme, in photosynthetic organisms. The creation of plants and other photosynthetic organisms having improved Rubisco biosynthetic pathways can provide increased yields of certain types of foodstuffs, enhanced biomass energy sources, and may alter the types and amounts of nutrients present in certain foodstuffs, among other desirable phenotypes.

Thus, there exists a need for improved methods for producing plants and agricultural photosynthetic microbes with an improved Rubisco enzyme. In particular, these methods should provide general means for producing novel Rubisco enzymes, including increasing the diversity of the Rubisco gene pool and the rate at which genetic sequences encoding one or more Rubisco subunits having desired properties are evolved. It is particularly desirable to have methods which are suitable for rapid evolution of genetic sequences to function in one or more plant species and confer an improved Rubisco phenotype (e.g., reduced sensitivity to atmospheric oxygen, increased carboxylation rate) to plants which express the genetic sequence(s).

The present invention meets these and other needs and provides such improvements and opportunities.

The references discussed herein are provided solely for their disclosure prior to the filing date of the present application. Nothing herein is to be construed as an admission that the inventors are not entitled to antedate such disclosure by virtue of prior invention. All publications cited are incorporated herein by reference, whether specifically noted as such or not.

### SUMMARY OF THE INVENTION

In a broad general aspect, the present invention provides a method for the rapid evolution of polynucleotide sequences encoding a Rubisco enzyme, or subunit thereof, that, when transferred into an appropriate plant cell, or photosynthetic microbial host and expressed therein, confers an enhanced metabolic phenotype to the host to increase carbon fixation efficiency and/or rate, or to increase the accumulation or depletion of certain metabolites. In general, polynucleotide sequence shuffling and phenotype selection, such as detection of a parameter of Rubisco enzyme activity, is employed recursively to generate polynucleotide sequences which encode novel proteins having desirable Rubisco enzymatic catalytic function(s), regulatory function(s), and related enzymatic and physicochemical properties. Although the method is believed broadly applicable to evolving biosynthetic enzymes having desired properties, the invention is described principally with reference to the metabolic enzyme activities of plants and/or photosynthetic microbes defined as ribulose-1,5-bisphosphate carboxylase/oxygenase (“Rubisco”), including both regulatory subunit (small subunit, S; gene designation, rbcS) and catalytic subunit (large subunit, L; gene designation, rbcL), respectively, as appropriate for Form I (L₈S₈) and Form II (L₂) Rubisco.

### Rubisco Embodiment—Lowered Km for CO₂

The invention provides an isolated polynucleotide encoding an enhanced rubisco protein having Rubisco catalytic activity wherein the Km for CO₂ is significantly lower than a protein encoded by a parental polynucleotide encoding a naturally-occurring Rubisco enzyme. Typically, the Km for CO₂ will be at least one-half logarithm unit lower than the parental sequence, preferably the Km will be at least one logarithm unit lower, and desirably the Km will be at least two logarithm units lower, or more. The isolated polynucleotide encoding an enhanced Rubisco protein and in an expressible form can be transferred into a host plant, such as a crop species, wherein suitable expression of the polynucleotide in the host plant results in improved carbon fixation efficiency as compared to the naturally-occurring host plant species, usually under certain atmospheric conditions. The isolated polynucleotide can encode a single subunit Rubisco, such as a Form II bacterial form, or may encode a large (L) subunit or small (S) subunit of a multisubunit Form I Rubisco such as that found in cynaobacteria, green algae, and higher plants. The isolated polynucleotide can comprise a substantially full-length or full-length coding sequence substantially identical to a naturally occurring rbcS gene and/or an rbcL gene, typically comprising a shuffled rbcL gene or a shuffled rbcL gene, or both.

In a variation, the invention provides a polynucleotide comprising: (1) a sequence encoding a shuffled Rubisco Form I L subunit gene (rbcL) linked to (2) a selectable marker gene which affords a means of selection when expressed in chloroplasts, and, optionally, flanked by (3) an upstream flanking recombinogenic sequence having sufficient sequence identity to a chloroplast genome sequence to mediate efficient recombination and (4) a downstream flanking recombinogenic sequence having sufficient sequence identity to a chloroplast genome sequence to mediate efficient recombination.

In a variation, the invention provides an isolated polynucleotide encoding an enhanced Rubisco protein having Rubisco catalytic activity wherein the Km for O₂ is significantly higher than a protein encoded by a parental polynucleotide encoding a naturally-occurring Rubisco enzyme or subunit. In an aspect, the enhanced Rubisco protein is often a L subunit which is catalytically active in the presence of a complementing S subunit. In an aspect, the enhanced Rubisco protein is a L subunit which is catalytically active in the absence of a complementing S subunit, such as for example and not limitation a Rubisco L subunit which is at least 90 percent sequence identical to a naturally occurring Form II L subunit.

In a variation, the invention provides an isolated polynucleotide encoding an enhanced Rubisco protein having Rubisco catalytic activity wherein the ratio of the Km for CO₂ to the Km for O₂ is significantly lower than a protein encoded by a parental polynucleotide encoding a naturally-occurring Rubisco enzyme.

The invention provides an enhanced Rubisco protein having Rubisco catalytic activity wherein: (1) the Km for CO₂ is significantly lower than a protein encoded by a parental polynucleotide encoding a naturally-occurring Rubisco enzyme, (2) the Km for O₂ is significantly higher than a protein encoded by a parental polynucleotide encoding a naturally-occurring Rubisco enzyme, and/or (3) the ratio of the Km for CO₂ to the Km for O₂ is significantly lower than a protein encoded by a parental polynucleotide encoding a naturally-occurring Rubisco enzyme.

Polynucleotide sequences encoding, e.g., a shuffled L subunit of a Form I hexadecimeric Rubisco are provided, where the shuffled L subunit possesses a detectable enzymatic activity wherein: (1) the Km for CO₂ is significantly lower than a L subunit protein encoded by a parental polynucleotide encoding a naturally-occurring Rubisco enzyme, (2) the Km for O₂ is significantly higher than an L subunit protein encoded by a parental polynucleotide encoding a naturally-occurring Rubisco enzyme, and/or (3) the ratio of the Km for CO₂ to the Km for O₂ is significantly lower than a L subunit protein encoded by a parental polynucleotide encoding a naturally-occurring Rubisco enzyme L subunit. In a variation, the shuffled L subunit requires a complementing S subunit for detectable enzymatic activity, or for increased enzymatic activity as compared to the activity of the shuffled L subunit in the absence of a complementing S subunit.

In an aspect, the invention provides a polynucleotide sequence encoding a shuffled S subunit of a Form I hexadecimeric Rubisco, wherein the shuffled S subunit possesses the property of complexing with an unshuffled, complementing L subunit thereby resulting in a multimer (e.g., hexadecimeric L₈S₈) having a detectable enzymatic activity wherein: (1) the Km for CO₂ is significantly lower than that of a Rubisco protein containing an S subunit encoded by a parental polynucleotide encoding a naturally-occurring S subunit of Rubisco, (2) the Km for O₂ is significantly higher than that of a Rubisco protein containing an S subunit encoded by a parental polynucleotide encoding a naturally-occurring S subunit of Rubisco, and/or (3) the ratio of the Km for CO₂ to the Km for O₂ is significantly lower than that of a Rubisco protein containing an S subunit encoded by a parental polynucleotide encoding a naturally-occurring S subunit of Rubisco.

An improved L subunit of a Form I Rubisco, or shufflant thereof, and a polynucleotide encoding the same are provided. In some embodiments, the polynucleotide is operably linked to a transcription regulation sequence forming an expression construct, which may be linked to a selectable marker gene. In some embodiments, such a polynucleotide is present as an integrated transgene in a plant chromosome, or more typically on a chloroplast chromosome in a format for expression and processing of the Form I L subunit in chloroplasts, which may be accomplished by homologous recombination targeting into a chloroplast genome. It can be desirable for such a polynucleotide transgene to be transmissible via germline transmission in a plant; in the case of rbcL sequences transferred to chloroplasts, it is often accompanied by a selectable marker gene which affords a means to select for progeny which retain chloroplasts having the transferred rbcL shuffled sequence. In an aspect, the invention provides an improved S subunit of a Form I Rubisco, or shufflant thereof, and a polynucleotide encoding same. In some embodiments, the polynucleotide will be operably linked to a transcription regulation sequence forming an expression construct, which may be linked to a selectable marker gene. In some embodiments, such a polynucleotide is present as an integrated transgene in a plant chromosome. It can be desirable for such a polynucleotide transgene to be transmissible via germline transmission in a plant.

In an aspect, the invention provides an improved L subunit of a Form II Rubisco, or shufflant thereof, and a polynucleotide encoding same. In some embodiments, the polynucleotide will be operably linked to a transcription regulation sequence forming an expression construct, which may be linked to a selectable marker gene. In some embodiments, such a polynucleotide is present as an integrated transgene in a plant chromosome. It can be desirable for such a polynucleotide transgene to be transmissible via germline transmission in a plant.

In an aspect, the invention provides a hybrid L subunit composed of a shufflant comprising a sequence of at least 25 contiguous nucleotides at least 95 percent identical to a Form I Rubisco rbcL gene and a sequence of at least 25 contiguous nucleotides at least 95 percent identical to a Form II Rubisco rbcL gene, and a polynucleotide encoding same, and typically encoding a substantially full-length Rubisco L subunit protein, usually comprising at least 90 percent of the coding sequence length, but not necessarily sequence identity, of a naturally occurring Rubisco L protein. In some embodiments, the polynucleotide will be operably linked to a transcription regulation sequence forming an expression construct, which may be linked to a selectable marker gene. In some embodiments, such a polynucleotide is present as an integrated transgene in a plant chromosome. It can be desirable for such a polynucleotide transgene to be transmissible via germline transmission in a plant.

The invention provides expression constructs, including plant transgenes, wherein the expression construct comprises a transcriptional regulatory sequence functional in plants operably linked to a polynucleotide encoding an enhanced Rubisco protein subunit. With respect to polynucleotide sequences encoding Form I Rubisco L subunit proteins, it is generally desirable to express such encoding sequences in plastids, such as chloroplasts, for appropriate transcription, translation, and processing. The invention further provides plants and plant germplasm comprising said expression constructs, typically in stably integrated or other replicable form which segregates and can be stably maintained in the host organism, although in some embodiments it is desirable for commercial reasons that the expression sequence not be in the germline of sexually reproducible plants.

The invention provides a method for obtaining an isolated polynucleotide encoding an enhanced Rubisco protein having Rubisco catalytic activity wherein the Km for CO₂ is significantly lower than a protein encoded by a parental polynucleotide encoding a naturally-occurring Rubisco enzyme, the method comprising: (1) recombining sequences of a plurality of parental polynucleotide species encoding at least one Rubsico sequence under conditions suitable for sequence shuffling to form a resultant library of sequence-shuffled Rubisco polynucleotides, (2) transferring said library into a plurality of host cells forming a library of transformants wherein sequence-shuffled Rubisco polynucleotides are expressed, (3) assaying individual or pooled transformants for Rubisco catalytic activity to determine the relative or absolute Km for CO₂ and identifying at least one enhanced transformant that expresses a Rubisco activity which has a significantly lower Km for CO₂ than the Rubisco activity encoded by the parental sequence(s), (4) recovering the sequence-shuffled Rubisco polynucleotide from at least one enhanced transformant. Optionally, the recovered sequence-shuffled Rubisco polynucleotide encoding an enhanced Rubisco is recursively shuffled and selected by repeating steps 1 through 4, wherein the recovered sequence-shuffled Rubisco polynucleotide is used as at least one parental sequence for subsequent shuffling. If it is desired to obtain a sequence-shuffled Rubisco encoding a Rubisco enzyme having an increased Km for O₂, step 3 comprises assaying individual or pooled transformants for Rubisco catalytic activity to determine the relative or absolute Km for O₂ and identifying at least one enhanced transformant that expresses a Rubisco activity which has a significantly higher Km for O₂ than the Rubisco activity encoded by the parental sequence(s). Similarly, if it is desired to obtain a sequence-shuffled Rubisco encoding a Rubisco enzyme having a decreased ratio of Km for CO₂ to Km for O₂, step 3 comprises assaying individual or pooled transformants for Rubisco catalytic activity to determine the relative or absolute Km for O₂ and Km for CO₂ identifying at least one enhanced transformant that expresses a Rubisco activity which has a significantly lower ratio of Km for CO₂ to Km for O₂ than the Rubisco activity encoded by the parental sequence(s).

In an aspect, the method is used to generate sequence-shuffled Rubisco polynucleotides encoding a single subunit Rubisco which is catalytically active in the absence of heterologous proteins. For example and not limitation, a bacterial single subunit Rubisco gene, such as that from Rhodospirillum rubrum (Falcone et al. (1993) J. Bacteriol. 175: 5066) is obtained as an isolated polynucleotide and is shuffled by any suitable shuffling method known in the art, such as DNA fragmentation and PCR, error-prone PCR, and the like, preferably with one or more additional parental polynucleotides encoding all or a part of another Rubisco species, which may be a single subunit Rubisco, or one subunit of a multisubunit Rubisco, such as a plant or cyanobacterial Rubisco L or S subunit. The population of sequence-shuffled Rubisco polynucleotides are each operably linked to an expression sequence and transferred into host cells, preferably host cells substantially lacking endogenous Rubisco activity, such as a deletion strain of Rhodospirillum rubrum Rubisco deletion strain (Falcone et al. op.cit), wherein the sequence-shuffled Rubisco polynucleotides are expressed, forming a library of sequence-shuffled Rubisco transformants. A sample of individual transformants and/or their clonal progeny are isolated into discrete reaction vessels for Rubisco activity assay, or are assayed in situ in certain embodiments. For samples assayed in reaction vessels, aliquots of the samples are separated into a plurality of reaction vessels containing an approximately equimolar amount of Rubisco or total protein, and each vessel is assayed for carboxylase activity in the presence of a predetermined concentration of CO₂ which ranges from about 0.0001 times the predetermined Km for CO₂ of the Rubisco encoded by the parental polynucleotide(s) to about 10,000 times the predetermined Km for CO₂ of the Rubisco encoded by the parental polynucleotide(s). From the data generated by assaying the plurality of reaction vessels containing aliquots of each transformant, a Km value is calculated by conventional art-known means for the sequence-shuffled Rubisco of each transformant. Sequence-shuffled polynucleotides encoding Rubisco proteins that have significantly decreased Km values for CO₂ are selected and used as parental sequences for at least one additional round of sequence shuffling by any suitable method and selection for decreased Km values for CO₂. The shuffling and selection process is performed iteratively until sequence shuffled polynucleotides encoding at least one Rubisco enzyme having a desired Km value is obtained, or until the optimization to reduce the Km has plateaued and no further improvement is seen in subsequent rounds of shuffling and selection.

In a variation, the sequence-shuffled polynucleotides operably linked to an expression sequence is also linked, in polynucleotide linkage, to an expression cassette encoding a selectable marker gene. Transformants are propagated on a selective medium to ensure that transformants which are assayed for Rubisco carboxylase activity contain a sequence-shuffled Rubisco encoding sequence in expressible form. In embodiments wherein a polynucleotide encoding an L subunit are to be introduced into host cells which possess chloroplasts, the L subunit encoding sequence is generally operably linked to a transcriptional regulatory sequence functional in chloroplasts and the resultant expression cassette is transferred into the host cell chloroplasts, such as by biolistics, polyethylene glycol (PEG) treatment of protoplasts, or an other suitable method.

In a variation, the above-described method is modified such that Rubisco oxygenase activity is assayed in the presence of varying concentrations of oxygen and the Km for O₂ is determined. Each vessel containing an aliquot of a transformant is assayed for oxygenase activity in the presence of a predetermined concentration of O₂ which ranges from about 0.0001 times the predetermined Km for O₂ of the Rubisco encoded by the parental polynucleotide(s) to about 10,000 times the predetermined Km for O₂ of the Rubisco encoded by the parental polynucleotide(s). From the data generated by assaying the plurality of reaction vessels containing aliquots of each transformant, a Km value is calculated by conventional art-known means for the sequence-shuffled Rubisco of each transformant. Sequence-shuffled polynucleotides encoding Rubisco proteins that have significantly increased Km values for O₂ are selected and used as parental sequences for at least one additional round of sequence shuffling by any suitable method and selection for decreased Km values for O₂. The shuffling and selection process is performed iteratively until sequence shuffled polynucleotides encoding at least one Rubisco enzyme having a desired Km value is obtained, or until the optimization to increase the Km has plateaued and no further improvement is seen in subsequent rounds of shuffling and selector.

In a variation, the method comprises conducting biochemical assays on sample aliquots of transformants to determine Rubisco enzyme activity so as to establish the ratio of the Km for CO₂ to the Km for O₂ for individual transformants. Sequence-shuffled polynucleotides encoding Rubisco are obtained from transformants exhibiting a decrease in said ratio as compared to the ratio in a Rubisco produced from the parental encoding polynucleotide(s) to provide selected sequence-shuffled Rubisco polynucleotides which can be used as parental sequences for at least one additional round of sequence shuffling by any suitable method and selection for a decreased ratio of Km(CO2) to Km(O2). The shuffling and selection process is performed iteratively until sequence shuffled polynucleotides encoding at least one Rubisco enzyme having a desired Km ratio is obtained, or until the optimization to decrease the Km ratio has plateaued and no further improvement is seen in subsequent rounds of shuffling and selection. Multiple rounds of recombination can be performed prior to any selection step to increase the diversity of resulting populations of nucleic acids prior to selection. Indeed, this approach can be used for recombination and selection processes indicated throughout this disclosure. Optionally, the host cell for transformation with sequence-shuffled polynucleotides encoding Rubisco is a Synechocystis mutant which lacks a Rubisco subunit protein, such as Synechocystis PCC6803, a mutant Rhodospirillum rubrum, or an equivalent.

In an embodiment of the method, the host cell comprises a cell expressing a complementing subunit of Rubisco which is capable of interacting with a Rubisco protein encoded by sequence-shuffled polypeptides encoding a Rubisco subunit. For example, if the shuffled polynucleotides encode a large subunit of Rubisco, a host cell for the transformation may endogenously encode a small subunit of Rubisco that may interact with a functional large subunit encoded by the shuffled polynucleotides. It is often desirable that such host cells lack expression of the endogenous Rubisco subunit corresponding to (e.g., cognate to) the type of subunit encoded by the shuffled polynucleotides. Mutant cell lines are available in the art and novel mutant Rubisco-deficient cells can be obtained by selecting from a pool of mutagenized cells those mutants which have lost detectable Rubisco activity, or by homologous gene targeting of rbcL and/or rbcS genes.

In an embodiment of the method, polynucleotides encoding naturally-occurring Rubisco protein sequences of a plurality of species of photosynthetic prokaryotes and/or dinoflagellates are shuffled by a suitable shuffling method to generate a shuffled Rubisco polynucleotide library, wherein each shuffled Rubisco encoding sequence is operably linked to an expression sequence, and which may optionally comprise a linked selectable marker gene cassette. Said library is transformed into Rhodosporillum or other photosynthetic bacteria which lack endogenous Rubisco activity, such as a Cbb⁻ mutant to form a transformed host cell library. The transformed host cell library is propagated on growth medium, which may contain a selection agent to ensure retention of a linked selectable marker gene, if present, but which requires carbon fixation form atmospheric CO₂ for cell propagation. The transformed host cell library is subjected to selection by incubating the cells under a graded range of concentrations of either: (1) CO₂ and inert gas, at decreasing concentrations of CO₂ to preferentially support growth of shufflants encoding Rubisco with a lower Km for CO₂; (2) CO₂, O₂ and inert gas, at increasing ratios of O₂/CO₂ to preferentially support growth of transformant cells expressing shufflants encoding relatively oxygen-insensitive Rubisco carboxlase activity, and/or (3) in CO₂, O₂, and inert gas of fixed concentration but at increasing temperature to select for shufflants encoding Rubisco with a lower Km for CO₂ and/or a higher Km for O₂. Transformed host cells which grow most robustly under the most stringent selection conditions that support growth are isolated individually or in pools, and the sequence-shuffled polynucleotide sequences encoding Rubisco are recovered, and optionally subjected to at least one subsequent iteration of shuffling and selection on growth medium, optionally using lower ranges of CO₂ concentration and/or higher ranges of O₂ concentration and/or higher temperature ranges for the selection step. The recovered sequence-shuffled Rubisco polynucleotide(s) encode(s) an enhanced Rubisco subunit protein.

In an embodiment of the method, a host cell comprising a non-photosynthetic bacterium, such as E. coli, lacking an endogenous ribulose-5-phosphate kinase activity, is transformed with an expression cassette encoding the production of a functional ribulose-5-phosphate kinase (“R5PK”) activity, thereby forming an R5PK host cell. R5PK encoding sequences are selected by the skilled artisan from publicly available sources. The method comprises transforming a population of R5PK host cells with a library of Rubisco polynucleotides, each Rubisco polynucleotide encoding a species of a shuffled Rubisco L subunit operably linked to a transcriptional control sequence forming an L subunit expression cassette, optionally including an expression cassette encoding a complementing Rubisco S subunit, culturing the population of transformed R5P host cells in the presence of labeled carbon dioxide (e.g., ¹⁴CO₂) and/or labeled bicarbonate for a suitable incubation period, determining the amount of labeled carbon that is fixed by each transformed host cell and its clonal progeny relative to the amount of carbon fixed by untransformed R5PK host cells cultured under equivalent conditions, including culture medium, atmosphere, incubation time and temperature, and selecting from said population of transformed R5PK host cells and their clonal progeny cells which exhibit labeled carbon fixation at statistically significant increased amount relative to said untransformed R5PK host cells, and segregating or isolating said selected transformed R5PK cells thereby forming a selected subpopulation of host cells harboring selected shuffled polynucleotides encoding Rubisco L subunit protein species having enhanced catalytic ability to fix carbon; said selected shuffled polynucleotides can be recovered and optionally subjected to additional rounds of shuffling and selection for enhanced carbon fixation to provide one or more optimized shuffled L subunit encoding sequences. The method may be modified for selecting optimized shuffled S subunit encoding polynucleotides; in this variation the R5PK host cells harbor expression cassettes encoding a complementing L subunit and the library comprises shuffled S subunit encoding sequences. In embodiments wherein host cells are non-photosynthetic bacteria, the Rubisco encoding sequences are generally substantially identical to naturally-occurring Form II L subunit sequences and/or cyanobacterial L subunit sequences, so as to ensure proper function in a prokaryotic host. In a variation, the transformed R5PK host cells are segregated in culture vessels, such as a multimicrowell plate, wherein each vessel comprises a subpopulation of species of transformed R5PK host cells and their clonal progeny, often consisting of a single species of transformed R5PK host cell and its clonal progeny, if any. Typically, the expression cassettes encoding the shuffled Rubisco subunit proteins are linked to a selectable marker gene cassette and selection is applied, typically by selection with an antibiotic in the culture medium, to reduce the prevalence of untransformed R5PK cells.

The invention provides a variation of the R5PK host cell method, wherein the host cell is a strain of non-photosynthetic bacterium which lacks endogenous phosphoglycerate kinase (PGK) activity; such a strain of E. coli is available from American Type Culture Collection, Rockville, Md. (Irani et al. (1977) J. Bacteriol. 132: 398). In this variation, the PGK⁻ host cell harbors an expression cassette encoding R5P kinase (R5PK) forming a PGK(−)/R5PK host cell. A population of PGK(−)/R5PK host cells are transformed with library members encoding the expression of shuffled Rubisco L (or S) subunits, optionally also encoding a complementing subunit if appropriate, culturing the population of transformed R5PK host cells in a minimal growth medium including glucose, wherein the minimal medium including glucose is insufficient to support the growth and replication of an untransformed PGK−/R5PK host cell, but is sufficient to support the growth and replication of a transformed PGK−/R5PK host cell expressing a functional Rubisco carboxylase activity. Transformed host cells are cultured in the minimal medium with glucose for a suitable incubation period and those transformed cells which express Rubisco carboxylase activity grow in the minimal medium plus glucose and are thereby selected from the population of transformed host cells and untransformed host cells, each of which substantially lacks the capacity to grow and replicate on the medium. The transformed host cells which grow and replicate thereby form a selected subpopulation of host cells harboring selected shuffled polynucleotides encoding Rubisco L (or S) subunit protein species having enhanced catalytic ability to fix carbon; said selected shuffled polynucleotides can be recovered and optionally subjected to additional rounds of shuffling and selection for enhanced carbon fixation to provide one or more optimized shuffled L (or S) subunit encoding sequences. The method may be modified for selecting optimized shuffled S subunit encoding polynucleotides; in this variation the PGK−/RSPK host cells harbor expression cassettes encoding a complementing L subunit and the library comprises shuffled S subunit encoding sequences. In a variation, the transformed R5PK host cells are segregated in culture vessels, such as a multimicrowell plate, wherein each vessel comprises a subpopulation of species of transformed PGK−/R5PK host cells and their clonal progeny.

The invention provides a plant cell protoplast and clonal progeny thereof containing a sequence-shuffled polynucleotide encoding a Rubisco subunit which is not encoded by the naturally occurring genome of the plant cell protoplast. The invention also provides a collection of plant cell protoplasts transformed with a library of sequence-shuffled Rubisco subunit polynucleotides in expressible form. The invention further provides a plant cell protoplast co-transformed with at least two species of library members wherein a first species of library members comprise sequence-shuffled Rubisco large subunit polynucleotides and a second species of library members comprise sequence-shuffled Rubisco small subunit polynucleotides. Typically, the large subunit polynucleotides are transferred into a plastid compartment for expression and processing, such as by transfer into chloroplasts in a format suitable for expression in the plastid, such as for example and not limitation as a recombinogenic construct for general targeted recombination into a chloroplast chromosome. Typically, small subunit polynucleotides are transferred into the protoplast nucleus for expression, and, if desired, integration or homologous recombination (or gene replacement of the endogenous rbc gene(s)).

The invention also provides a regenerated plant containing at least one species of replicable or integrated polynucleotide comprising a sequence-shuffled portion and encoding a Rubisco subunit polypeptide. The invention provides a method variation wherein at least one round of phenotype selection is performed on regenerated plants derived from protoplasts transformed with sequence-shuffled Rubisco subunit library members.

The invention provides species-specific Rubisco shuffling, wherein a transformed plant cell or adult plant or reproductive structure comprises a polynucleotide encoding a shuffled Rubisco subunit that is at least 95 percent sequence identical to the corresponding Rubisco subunit encoded by an untransformed naturally-occurring genome of the same taxonomic species of plant cell or adult plant. Typically, the shuffled Rubisco subunit results from shuffling of one or more alleles encoding the Rubsico subunit in the taxonomic species genome, optionally including mutagenesis in one or more of the iterative shuffling and selection cycles. The species-specific Rubisco shuffling may include shuffling a polynucleotide encoding a full-length Rubisco subunit of a first taxonomic species under conditions whereby Rubisco subunit sequences of a second taxonomic species (or collection of species) are shuffled in at a low prevalence, such that the resultant population of shufflant polynucleotides contains, on average, shuffled polynucleotides composed of at least about 95 percent sequence encoding the first taxonomic species Rubisco subunit and less than about 5 percent sequence encoding the second taxonomic species (or collection of species) Rubsico subunit. The species-specific shufflants are thus highly biased towards identity with the first taxonomic species and shufflants which are selected for the desired Rubisco phenotype are transferred back into the first taxonoic species for expression and regeneration of adult plants and germplasm. Optionally, selected shufflants are backcrossed against the naturally occurring Rubisco encoding sequences of the first taxonomic species to and harmonize the final shufflant sequence to the naturally-occurring Rubisco sequence of the first taxonomic species.

An object of the invention is the production of higher plants which express one or more Rubsico enzyme subunits which confer an enhanced carbon fixation ratio (or net carbon fixation rate) to the plants. Although the invention is described principally with respect to the use of genetic sequence shuffling to generate enhanced Rubisco coding sequences, the invention also provides for the introduction of Rubisco coding sequences obtained from marine green algae, such as high specificity chromophytic and/or rhodophytic algae encoding Rubisco enzymes having ratios of K_{O2}/K_{CO2} greater than those ratios in terrestrial plant Rubisco species, into higher plants. Thus, the invention provides a method comprising the step of introducing into a higher plant (e.g., a monocot or dicot) an expression cassette encoding a Rubisco encoded by a genome of a marine algae; in preferred embodiments the marine algae are Porphyridium, Olisthodiscus, Cryptomonas, C. fusiformis, or Cylindrotheca N1. Typically, at least a sequence encoding a substantially full-length large subunit of the marine algal Rubisco is transferred; often a sequence encoding a substantially full-length small subunit of the marine algal Rubisco is also transferred. In some embodiments, the endogenous Rubisco encoded by the naturally-occurring higher plant genome (including the chloroplast genome encoding the L subunit) is functionally inactivated (e.g., often all such alleles present in the genome are disrupted to provide for homozygosity for the knockout of endogenous Rubisco) to reduce competition by endogenous Rubsico, however suppression of endogenous Rubisco may be accomplished by alternative methods including but not limited to sense suppression, antisense suppression, and other methods known in the art. An aspect of the invention provides C4 land plants comprising a polynucleotide sequence encoding a marine algal Rubsico, such as a polynucleotide encoding a Rubisco large subunit of Porphyridium or Cylindrotheca N1 composed in an expression cassette suitable for expression in chloroplasts of the C4 land plant; optionally an expression cassette encoding a complementing marine algal small subunit operably linked to regulatory sequences for expression in the nucleus of the C4 plant additionally is transferred into the nucleus of the C4 plant. The large subunit expression cassette is transferred into the chloroplasts of a regenerable plant cell (e.g. a protoplast of a C4 plant cell), and optionally the small subunit expression vector is transferred into the nucleus of the regenerable plant cell, both by art-known transformation methods. A C3 plant may be used in place of a C4 plant if desired. A specific embodiment comprises a regenerable protoplast of Glycine max, Nicotiana tabacum, or Zea mays (or other agricultural crop species amenable to regeneration from protoplasts) having a chloroplast genome containing an expressible Rubisco large subunit gene that is obtained from a marine algae, such as Porphyridium or Cylindrotheca N1, and typically is at least 98 percent up to 100 percent sequence identical to a Rubisco large subunit gene in the genome of said marine algae. The regenerable protoplast may further contain a nuclear genome containing an expressible Rubisco small subunit gene that is obtained from a marine algae, such as Porphyridium or Cylindrotheca N1, and typically is at least 98 percent up to 100 percent sequence identical to a Rubisco large subunit gene in the genome of said marine algae, and that is a complementing subunit of said marine algal large subunit. The invention also provides adult plants, cultivars, seeds, vegetative bodies, fruits, germplasm, and reproductive cells obtained from regeneration of such transformed protoplasts.

The invention provides a kit for obtaining a polynucleotide encoding a Rubisco protein, or subunit thereof, having a predetermined enzymatic phenotype, the kit comprising a cell line suitable for forming transformable host cells and a collection sequence-shuffled polynucleotides formed by in vitro sequence shuffling. The kit often further comprises a transformation enhancing agent (e.g., lipofection agent, PEG, etc.) and/or a transformation device (e.g., a biolistics gene gun) and/or a plant viral vector which can infect plant cells or protoplasts thereof.

The disclosed method for providing an agricultural organism having an improved Rubisco enzymatic phenotype by iterative gene shuffling and phenotype selection is a pioneering method which enables a broad range of novel and advantageous agricultural compositions, methods, kits, uses, plant cultivars, and apparatus which will be apparent to those skilled in the art in view of the present disclosure.

In one aspect, the invention provides methods of producing a recombinant cell having an elevated carbon fixation activity. In the methods, one or more first Calvin or Krebs cycle enzyme (e.g., rubisco) coding nucleic acid, or a homologue thereof, is recombined with one or more homologous first nucleic acid to produce a library of recombinant first enzyme nucleic acid homologues. This step can be repeated as desired to produce a more diverse library of recombinant first enzyme nucleic acid homologues. The libraries are selected for an activity which aids in carbon fixation, such as an increased catalytic rate, an altered substrate specificity, an increased ability of a cell expressing one or more members of the library to fix CO₂ when the one or more library members is expressed in the cell, etc., thereby producing a selected library of recombinant first enzyme nucleic acid homologues. These steps are recursively repeated until one or more members of the selected library produces an elevated carbon fixation level in a target recombinant cell when the one or more selected library member is expressed in the target cell, as compared to a carbon fixation activity of the target cell when the one or more selected library member is not expressed in the target cell.

Kits comprising the components herein and, optionally, instructions for practicing the methods herein, are a feature of the invention. Optionally, kits will further include, e.g., containers, packaging materials, etc. Further, integrated systems comprising sequences corresponding to any nucleic acid or polypeptide sequence as set forth herein, or as provided by the methods herein, are a feature of the invention.

Other features and advantages of the invention will be apparent from the following description of the drawings, preferred embodiments of the invention, the examples, and the claims.

### BRIEF DESCRIPTION OF THE DRAWINGS

FIG. 1. Shows a flow diagram for an embodiment for shuffling Form I Rubisco L subunit to improve carboxylation specificity.

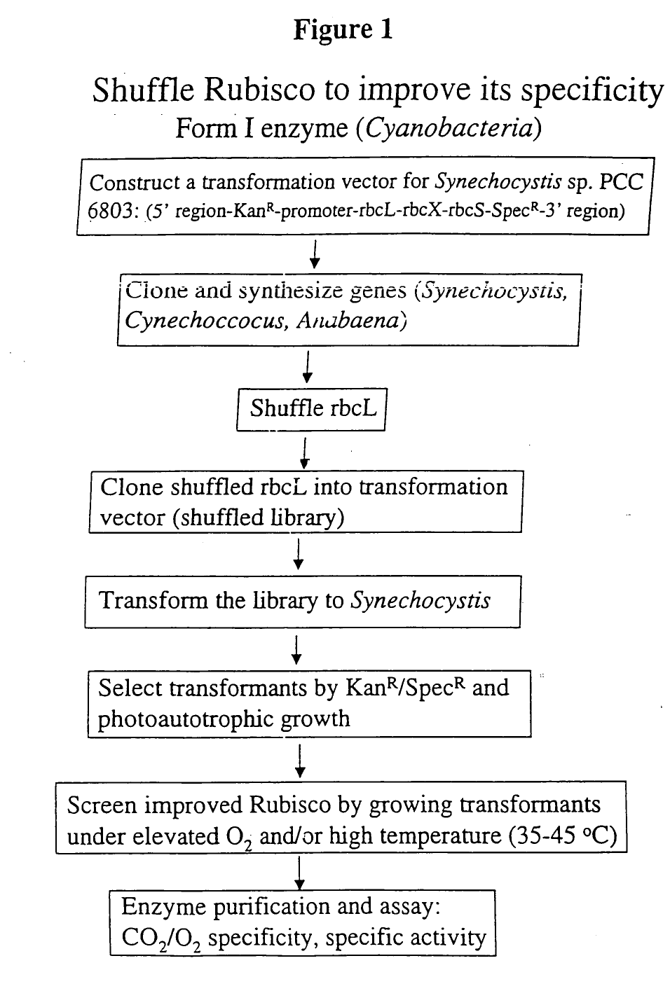

FIG. 2. (Panel A) Synechocystis Rubisco gene organization. (Panel B) Diagram showing homologous recombination method and constructs for replacing Synechocystis Rubisco rbcL gene.

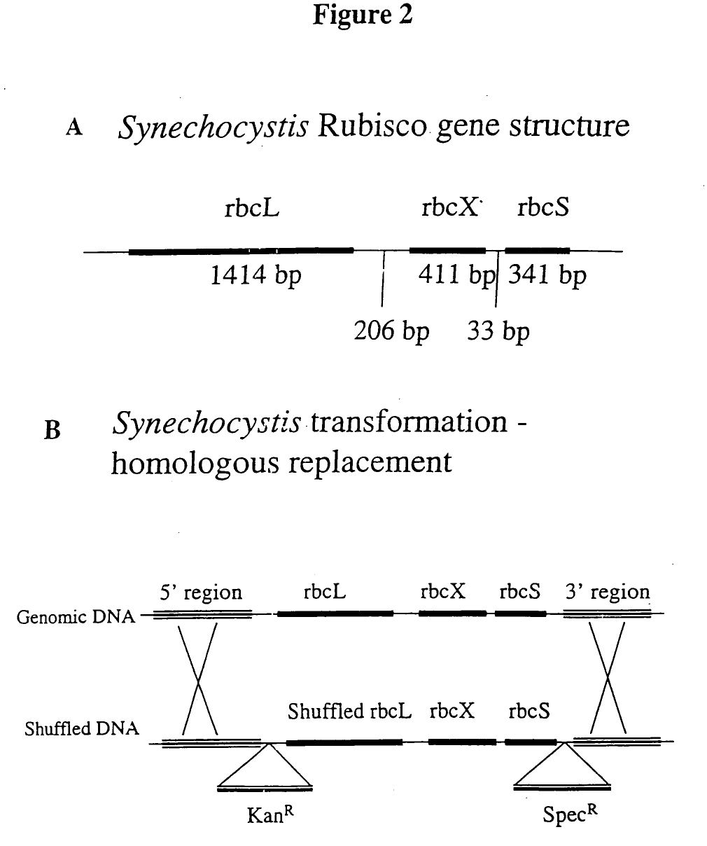

FIG. 3. Shows a flow diagram for an embodiment for shuffling Form II Rubisco L subunit to improve carboxylation specificity.

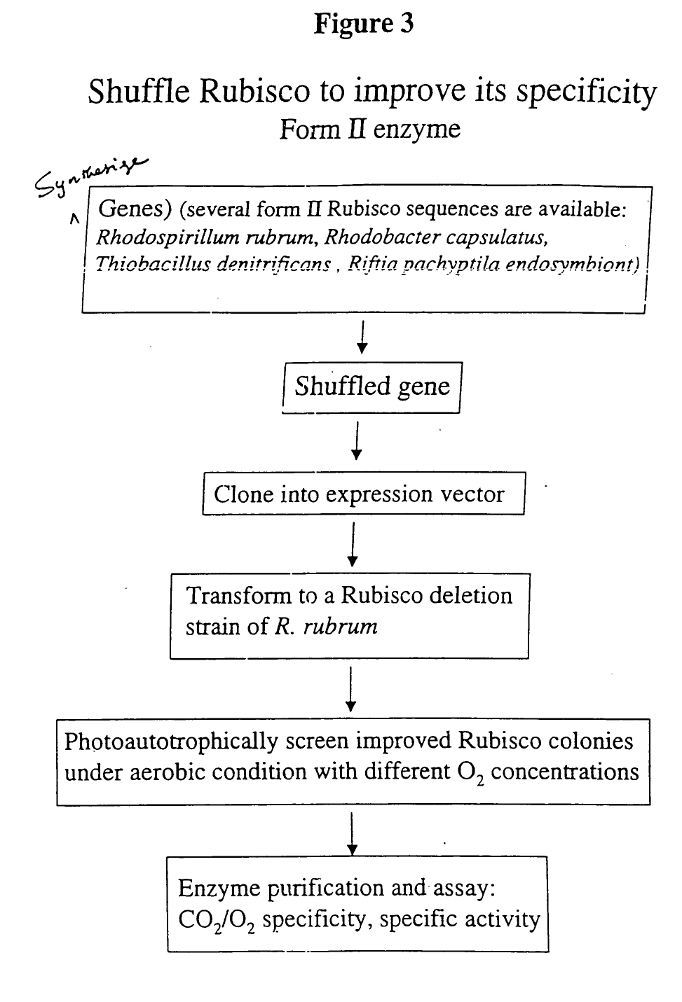

FIG. 4. Shows a flow diagram for an embodiment for shuffling Form II Rubisco L subunit to improve carboxylation specificity using PRK(−) host cells.

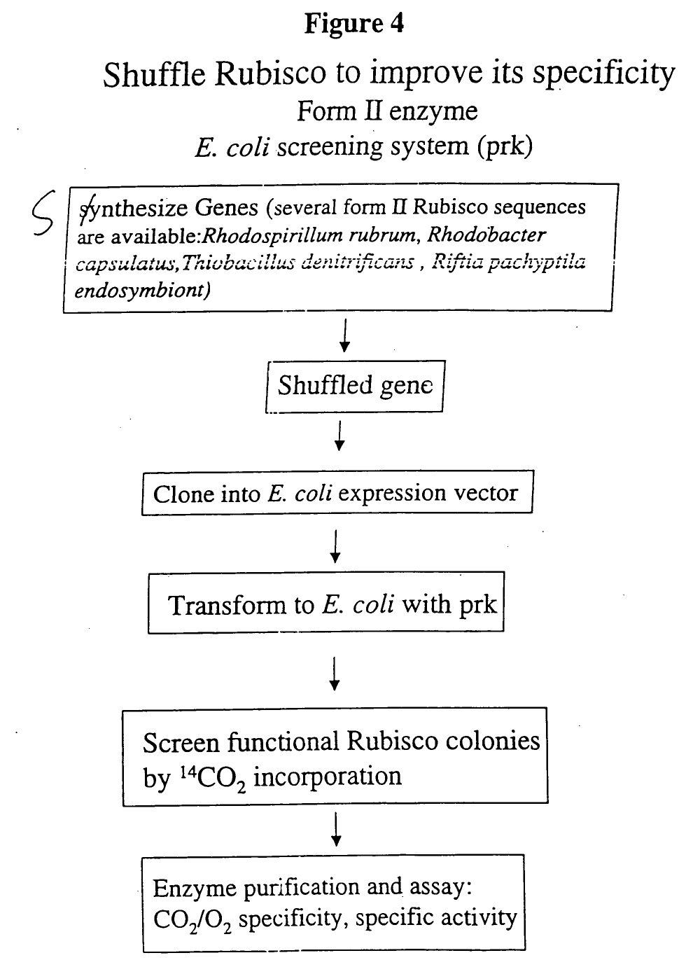

FIG. 5. Shows a flow diagram for an embodiment shuffling a Rubisco rbcL/S operon from high specificity marine algae.

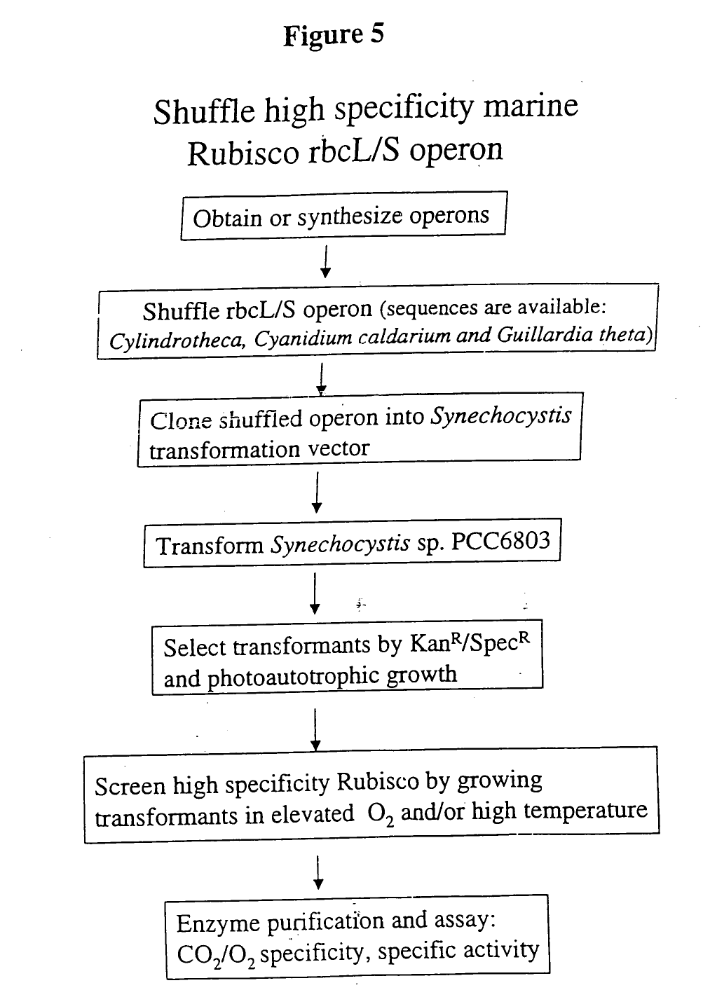

### DETAILED DESCRIPTION

Definitions

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are described. For purposes of the present invention, the following terms are defined below.

The term “shuffling” is used herein to indicate recombination between similar but non-identical polynucleotide sequences. Generally, more than one cycle of recombination is performed in DNA shuffling methods. In some embodiments, DNA shuffling may involve crossover via nonhomologous recombination, such as via cre/lox and/or flp/frt systems and the like, such that recombination need not require substantially homologous polynucleotide sequences. In silico and oligonucleotide mediated approaches also do not require similarity/homology. Homologous and non-homologous recombination formats can be used, and, in some embodiments, can generate molecular chimeras and/or molecular hybrids of substantially dissimilar sequences. Viral recombination systems, such as template-switching and the like can also be used to generate molecular chimeras and recombined genes, or portions thereof. A general description of shuffling is provided in commonly-assigned WO98/13487 and WO98/13485, both of which are incorporated herein in their entirety by reference; in case of any conflicting description of definition between any of the incorporated documents and the text of this specification, the present specification provides the principal basis for guidance and disclosure of the present invention.

The term “related polynucleotides” means that regions or areas of the polynucleotides are identical and regions or areas of the polynucleotides are heterologous.

The term “chimeric polynucleotide” means that the polynucleotide comprises regions which are wild-type and regions which are mutated. It may also mean that the polynucleotide comprises wild-type regions from one polynucleotide and wild-type regions from another related polynucleotide. The term “cleaving” means digesting the polynucleotide with enzymes or breaking the polynucleotide (e.g., by chemical or physical means), or generating partial length copies of a parent sequence(s) via partial PCR extension, PCR stuttering, differential fragment amplification, or other means of producing partial length copies of one or more parental sequences. A “fragmented population” of nucleic acids is produced by cleavage of a polynucleotide as indicated, or by producing oligonucleotide sets that correspond to one or more parental nucleic acid.

The term “population,” as used herein, means a collection of components such as polynucleotides, nucleic acid fragments, or proteins. A “mixed population” means a collection of components which belong to the same family of nucleic acids or proteins (i.e. are related) but which differ in their sequence (i.e. are not identical) and hence in their biological activity.

The term “mutations” means changes in the sequence of a parent nucleic acid sequence (e.g., a gene or a microbial genome, transferable element, or episome) or changes in the sequence of a parent polypeptide. Such mutations may be point mutations such as transitions or transversions. The mutations may be deletions, insertions or duplications.

The term “recursive sequence recombination” as used herein refers to a method whereby a population of polynucleotide sequences are recombined with each other by any suitable recombination means (e.g., sexual PCR, homologous recombination, site-specific recombination, etc.) to generate a library of sequence-recombined species which is then screened or subjected to selection to obtain those sequence-recombined species having a desired property; the selected species are then subjected to at least one additional cycle of recombination with themselves and/or with other polynucleotide species and at subsequent selection or screening for the desired property.

The term “amplification” means that the number of copies of a nucleic acid fragment is increased.

The term “naturally-occurring” as used herein as applied to an object refers to the fact that an object can be found in nature. For example, a polypeptide or polynucleotide sequence that is present in an organism that can be isolated from a source in nature and which has not been intentionally modified by man in the laboratory is naturally-occurring. As used herein, laboratory strains and established cultivars of plants which may have been selectively bred according to classical genetics are considered naturally-occurring. As used herein, naturally-occurring polynucleotide and polypeptide sequences are those sequences, including natural variants thereof, which can be found in a source in nature, or which are sufficiently similar to known natural sequences that a skilled artisan would recognize that the sequence could have arisen by natural mutation and recombination processes.

As used herein “predetermined” means that the cell type, non-human animal, or virus may be selected at the discretion of the practitioner on the basis of a known phenotype.

As used herein, “linked” means in polynucleotide linkage (i.e., phosphodiester linkage). “Unlinked” means not linked to another polynucleotide sequence; hence, two sequences are unlinked if each sequence has a free 5′ terminus and a free 3′ terminus.

As used herein, the term “operably linked” refers to a linkage of polynucleotide elements in a functional relationship. A nucleic acid is “operably linked” when it is placed into a functional relationship with another nucleic acid sequence. For instance, a promoter or enhancer is operably linked to a coding sequence if it affects the transcription of the coding sequence. Operably linked means that the DNA sequences being linked are typically contiguous and, where necessary to join two protein coding regions, contiguous and in reading frame. However, since enhancers generally function when separated from the promoter by several kilobases and intronic sequences may be of variable lengths, some polynucleotide elements may be operably linked but riot contiguous. A structural gene (e.g., a RUBISCO gene) which is operably linked to a polynucleotide sequence corresponding to a transcriptional regulatory sequence of an endogenous gene is generally expressed in substantially the same temporal and cell type-specific pattern as is the naturally-occurring gene.

As used herein, the terms “expression cassette” refers to a polynucleotide comprising a promoter sequence and, optionally, an enhancer and/or silencer element(s), operably linked to a structural sequence, such as a cDNA sequence or genomic DNA sequence. In some embodiments, an expression cassette may also include polyadenylation site sequences to ensure polyadenylation of transcripts. When an expression cassette is transferred into a suitable host cell, the structural sequence is transcribed from the expression cassette promoter, and a translatable message is generated, either directly or following appropriate RNA splicing. Typically, an expression cassette comprises: (1) a promoter, such as a CaMV 35S promoter, a NOS promoter or a rbcS promoter, or other suitable promoter known in the art, (2) a cloned polynucleotide sequence, such as a cDNA or genomic fragment ligated to the promoter in sense orientation so that transcription from the promoter will produce a RNA that encodes a functional protein, and (3) a polyadenylation sequence. For example and not limitation, an expression cassette of the invention may comprise the cDNA expression cloning vectors, pCD and λNMT (Okayama H and Berg P (1983) Mol. Cell. Biol. 3: 280; Okayama H and Berg P (1985) Mol. Cell. Biol. 5: 1136, incorporated herein by reference). With reference to expression cassettes which are designed to function in chloroplasts, such as an expression cassette encoding a large subunit of Rubisco (rbcL) in a higher plant, the expression cassette comprises the sequences necessary to ensure expression in chloroplasts—typically the Rubisco L subunit encoding sequence is flanked by two regions of homology to the plastid genome so as to effect a homologous recombination with the chloroplastid genome; often a selectable marker gene is also present within the flanking plastid DNA sequences to facilitate selection of genetically stable transformed chloroplasts in the resultant transplastonic plant cells (see Maliga P (1993) TIBTECH 11: 101; Daniell et al. (1998) Nature Biotechnology 16: 346, and references cited therein).

As used herein, the term “transcriptional unit” or “transcriptional complex” refers to a polynucleotide sequence that comprises a structural gene (exons), a cis-acting linked promoter and other cis-acting sequences necessary for efficient transcription of the structural sequences, distal regulatory elements necessary for appropriate tissue-specific and developmental transcription of the structural sequences, and additional cis sequences important for efficient transcription and translation (e.g., polyadenylation site, mRNA stability controlling sequences).

As used herein, the term “transcription regulatory region” refers to a DNA sequence comprising a functional promoter and any associated transcription elements (e.g., enhancer, CCAAT box, TATA box, LRE, ethanol-inducible element, etc.) that are essential for transcription of a polynucleotide sequence that is operably linked to the transcription regulatory region.

As used herein, the term “xenogeneic” is defined in relation to a recipient genome, host cell, or organism and means that an amino acid sequence or polynucleotide sequence is not encoded by or present in, respectively, the naturally-occurring genome of the recipient genome, host cell, or organism. Xenogenic DNA sequences are foreign DNA sequences. Further, a nucleic acid sequence that has been substantially mutated (e.g., by site directed mutagenesis) is xenogeneic with respect to the genome from which the sequence was originally derived, if the mutated sequence does not naturally occur in the genome.

The term “corresponds to” is used herein to mean that a polynucleotide sequence is homologous (i.e., identical) to all or a portion of a reference polynucleotide sequence, or that a polypeptide sequence is identical to a reference polypeptide sequence. In contradistinction, the term “complementary to” is used herein to mean that the complementary sequence is homologous to all or a portion of a reference polynucleotide sequence. For illustration, the nucleotide sequence “5′-TATAC” corresponds to a reference sequence “5′-TATAC” and is complementary to a reference sequence “5′-GTATA”.

The following terms are used to describe the sequence relationships between two or more polynucleotides: “reference sequence”, “comparison window”, “sequence identity”, “percentage of sequence identity”, and “substantial identity”. A “reference sequence” is a defined sequence used as a basis for a sequence comparison; a reference sequence may be a subset of a larger sequence, for example, as a segment of a full-length viral gene or virus genome. Generally, a reference sequence is at least 20 nucleotides in length, frequently at least 25 nucleotides in length, and often at least 50 nucleotides in length. Since two polynucleotides may each comprise (1) a sequence (i.e., a portion of the complete polynucleotide sequence) that is similar between the two polynucleotides, and (2) a sequence that is divergent between the two polynucleotides, sequence comparisons between two (or more) polynucleotides are typically performed by comparing sequences of the two polynucleotides over a “comparison window” to identify and compare local regions of sequence similarity.

A “comparison window”, as used herein, refers to a conceptual segment of at least 25 contiguous nucleotide positions wherein a polynucleotide sequence may be compared to a reference sequence of at least 25 contiguous nucleotides and wherein the portion of the polynucleotide sequence in the comparison window may comprise additions or deletions (i.e., gaps) of 20 percent or less as compared to the reference sequence (which for comparative purposes in this manner does not comprise additions or deletions) for optimal alignment of the two sequences. Optimal alignment of sequences for aligning a comparison window may be conducted by the local homology algorithm of Smith and Waterman (1981) Adv. Appl. Math. 2: 482, by the homology alignment algorithm of Needleman and Wunsch (1970) J. Mol. Biol. 48: 443, by the search for similarity method of Pearson and Lipman (1988) Proc. Natl. Acad. Sci. (U.S.A.) 85: 2444, by computerized implementations of these algorithms (GAP, BESTFIT, FASTA, and TFASTA in the Wisconsin Genetics Software Package Release 7.0, Genetics Computer Group, 575 Science Dr., Madison, Wis.), or by inspection, and the best alignment (i.e., resulting in the highest percentage of homology over the comparison window) generated by the various methods is selected.

The term “sequence identity” means that two polynucleotide sequences are identical (i.e., on a nucleotide-by-nucleotide basis) over the window of comparison. The term “percentage of sequence identity” is calculated by comparing two optimally aligned sequences over the window of comparison, determining the number of positions at which the identical nucleic acid base (e.g., A, T, C, G, U, or I) occurs in both sequences to yield the number of matched positions, dividing the number of matched positions by the total number of positions in the window of comparison (i.e., the window size), and multiplying the result by 100 to yield the percentage of sequence identity. The term “substantial identity” as used herein denotes a characteristic of a polynucleotide sequence, wherein the polynucleotide comprises a sequence that has at least 80 percent sequence identity, preferably at least 85 percent identity and often 89 to 95 percent sequence identity, more usually at least 99 percent sequence identity as compared to a reference sequence over a comparison window of at least 20 nucleotide positions, optionally over a window of at least 30-50 nucleotides, wherein the percentage of sequence identity is calculated by comparing the reference sequence to the polynucleotide sequence that may include deletions or additions which total 20 percent or less of the reference sequence over the window of comparison. The reference sequence may be a subset of a larger sequence.

Specific hybridization is defined herein as the formation, by hydrogen bonding or nucleotide (or nucleobase) bases, of hybrids between a probe polynucleotide (e.g., a polynucleotide of the invention and a specific target polynucleotide, wherein the probe preferentially hybridizes to the specific target such that, for example, a single band corresponding to, e.g., one or more of the RNA species of the gene (or specifically cleaved or processed RNA species) can be identified on a Northern blot of RNA prepared from a suitable source. Such hybrids may be completely or only partially base-paired. Polynucleotides of the invention which specifically hybridize to viral genome sequences may be prepared on the basis of the sequence data provided herein and available in the patent applications incorporated herein and scientific and patent publications noted above, and according to methods and thermodynamic principles known in the art and described in Sambrooke et al. et al., Molecular Cloning: A Laboratory Manual, 2nd Ed., (1989), Cold Spring Harbor, N.Y.; Berger and Kimmel, Methods in Enzymology, Volume 152, Guide to Molecular Cloning Techniques (1987), Academic Press, Inc., San Diego, Calif.; Goodspeed et al. (1989) Gene 76: 1; Dunn et al. (1989) J. Biol. Chem. 264: 13057, and Dunn et al. (1988) J. Biol. Chem. 263: 10878, which are each incorporated herein by reference.

“Physiological conditions” as used herein refers to temperature, pH, ionic strength, viscosity, and like biochemical parameters that are compatible with a viable plant organism or agricultural microorganism (e.g., Rhizobium, Agrobacterium, etc.), and/or that typically exist intracellularly in a viable cultured plant cell, particularly conditions existing in the nucleus of said cell. In general, in vitro physiological conditions can comprise 50-200 mM NaCl or KCl, pH 6.5-8.5, 20-45° C. and 0.001-10 mM divalent cation (e.g., Mg⁺⁺, Ca⁺⁺); preferably about 150 mM NaCl or KCl, pH 7.2-7.6, 5 mM divalent cation, and often include 0.01-1.0 percent nonspecific protein (e.g., BSA). A non-ionic detergent (Tween, NP-40, Triton X-100) can often be present, usually at about 0.001 to 2%, typically 0.05-0.2% (v/v). Particular aqueous conditions may be selected by the practitioner according to conventional methods. For general guidance, the following buffered aqueous conditions may be applicable: 10-250 mM NaCl, 5-50 mM Tris HCl, pH 5-8, with optional addition of divalent cation(s), metal chelators, nonionic detergents, membrane fractions, antifoam agents, and/or scintillants.

As used herein, the terms “label” or “labeled” refer to incorporation of a detectable marker, e.g., a radiolabeled amino acid or a recoverable label (e.g. biotinyl moieties that can be recovered by avidin or streptavidin). Recoverable labels can include covalently linked polynucleobase sequences that can be recovered by hybridization to a complementary sequence polynucleotide. Various methods of labeling polypeptides, PNAs, and polynucleotides are known in the art and may be used. Examples of labels include, but are not limited to, the following: radioisotopes (e.g., ³H, ¹⁴C, ³⁵S, ¹²⁵I, ¹³¹I), fluorescent or phosphorescent labels (e.g., FITC, rhodamine, lanthanide phosphors), enzymatic labels (e.g., horseradish peroxidase, β-galactosidase, luciferase, alkaline phosphatase), biotinyl groups, predetermined polypeptide epitopes recognized by a secondary reporter (e.g., leucine zipper pair sequences, binding sites for antibodies, transcriptional activator polypeptide, metal binding domains, epitope tags). In some embodiments, labels are attached by spacer arms of various lengths, e.g., to reduce potential steric hindrance.

As used herein, the term “statistically significant” means a result (i.e., an assay readout) that generally is at least two standard deviations above or below the mean of at least three separate determinations of a control assay readout and/or that is statistically significant as determined by Student's t-test or other art-accepted measure of statistical significance.

The term “transcriptional modulation” is used herein to refer to the capacity to either enhance transcription or inhibit transcription of a structural sequence linked in cis; such enhancement or inhibition may be contingent on the occurrence of a specific extent, such as stimulation with an inducer and/or may only be manifest in certain cell types.

The term “agent” is used herein to denote a chemical compound, a mixture of chemical compounds, a biological macromolecule, or an extract made from biological materials such as bacteria, plants, fungi, or animal cells or tissues. Agents are evaluated for potential activity as Rubisco inhibitors or allosteric effectors by inclusion in screening assays described hereinbelow.

As used herein, “substantially pure” means an object species is the predominant species present (i.e., on a molar basis it is more abundant than any other individual macromolecular species in the composition), and preferably a substantially purified fraction is a composition wherein the object species comprises at least about 50 percent (on a molar basis) of all macromolecular species present. Generally, a substantially pure composition will comprise more than about 80 to 90 percent of all macromolecular species present in the composition. Most preferably, the object species is purified to essential homogeneity (contaminant species cannot be detected in the composition by conventional detection methods) wherein the composition consists essentially of a single macromolecular species. Solvent species, small molecules (<500 Daltons), and elemental ion species are not considered macromolecular species.

As used herein, the term “optimized” is used to mean substantially improved in a desired structure or function relative to an initial starting condition, not necessarily the optimal structure or function which could be obtained if all possible combinatorial variants could be made and evaluated, a condition which is typically impractical due to the number of possible combinations and permutations in polynucleotide sequences of significant length (e.g., a complete plant gene or genome).

As used herein, “Rubisco enzymatic phenotype” means an observable or otherwise detectable phenotype that can be discriminative based on Rubisco function. For example and not limitation, a Rubisco enzymatic phenotype can comprise an enzyme Km, for a substrate, VO2, VCO2, V_{O2}/V_{CO2}, (V_{CO2}K_{O2}/V_{O2}K_{CO2}) K_{RuBP}, a turnover rate, an inhibition coefficient (Ki), or an observable or otherwise detectable trait that reports Rubisco function in a cell or clonal progeny thereof which otherwise lack said trait in the absence of significant Rubisco function.

As used herein, “complementing subunit” is used principally with, reference to Form I Rubisco composed of S and I, subunits and means a Rubisco subunit of the opposite type (e.g., an S subunit can be a complementing subunit to an L subunit, and vice versa), wherein when the L and S subunits are present in a cell or in vitro reaction vessel under appropriate assay conditions they form a multimer having detectable Rubisco carboxylase activity. A complementing subunit can be obtained from the same taxonomic species of organism, or from a xenogenic species. Calibration assays are performed to determine whether a selected first subunit is a complementing subunit with respect to a second subunit; if the first subunit produces a detectable allosteric effect upon the activity, it is deemed for purposes of this disclosure to constitute a complementing subunit.

### DESCRIPTION OF PREFERRED EMBODIMENTS

The present invention provides methods, reagents, genetically modified plants, plant cells and protoplasts thereof, microbes, and polynucleotides, and compositions relating to the forced evolution of Rubisco subunit sequences to improve an enzymatic property of a Rubisco protein. In an aspect, the invention provides a shuffled Rubisco L subunit which is catalytically active in the presence of a complementing S subunit, which may itself be shuffled, and which exhibits an improved enzymatic profile, such as an increased Km for O₂, a decreased Km for CO₂, increased turnover rate for fixation of carbon, or the like. In an aspect, the shuffled L subunit is catalytically active in the absence of an S subunit and the presence of an S subunit does not significantly increase the catalytic activity of the L subunit as measured by RuBP carboxylase and/or RuBP oxygenase activity.

In a broad aspect, the invention is based, in part, on a method for shuffling polynucleotide sequences that encode a Rubisco subunit, such as a Form I rbcS subunit, a Form I rbcL subunit, or a Form II rbcL subunit, or combinations thereof. The method comprises the step of selecting at least one polynucleotide sequence that encodes a Rubisco subunit having an enhanced enzymatic phenotype and subjecting said selected polynucleotide sequence to at least one subsequent round of mutagenesis and/or sequence shuffling, and selection for the enhanced phenotype. Preferably, the method is performed recursively on a collection of selected polynucleotide sequences encoding the Rubisco subunit to iteratively provide polynucleotide sequences encoding Rubisco subunit species having the desired enhanced enzymatic phenotype.

The invention provides shuffled rbcL encoding sequences, wherein said shuffled encoding sequences comprise at least 21 contiguous nucleotides, preferably at least 30 contiguous nucleotides, or more, of a first naturally occurring rbcL gene sequence and at least 21 contiguous nucleotides, preferably at least 30 contiguous nucleotides, or more, of a second naturally occurring rbcL gene sequence, operably linked in reading frame to encode a Rubisco L subunit which has RuBP carboxylase activity in the presence of a complementing S subunit and/or in the absence of said S subunit, and which has an enhanced enzymatic phenotype. In some variations, it will be possible to use shuffled encoding sequences which have less than 21 contiguous nucleotides identical to a naturally-occurring rbcL gene sequence.

The invention also provides shuffled rbcS encoding sequences, wherein said shuffled encoding sequences comprise at least 21 contiguous nucleotides, preferably at least 30 contiguous nucleotides, or more, of a first naturally occurring rbcS gene sequence and at least 21 contiguous nucleotides, preferably at least 30 contiguous nucleotides, or more, of a second naturally occurring rbcL gene sequence, operably linked in reading frame to encode a Rubisco S subunit which has a regulatory effect upon a complementing Rubisco L subunit such that the multimer composed of the shuffled S subunit(s) and the L subunit(s) exhibit RuBP carboxylase activity and wherein the multimer has an enhanced enzymatic phenotype. In some variations, it will be possible to use shuffled encoding sequences which have less than 21 contiguous nucleotides identical to a naturally-occurring rbcS gene sequence.

The invention provides shuffled rbcL encoding sequences, wherein the shuffled sequences comprise portions of a first parental rbcL encoding sequence which comprises at least one mutation in the encoding sequence as compared to the collection of predetermined naturally occurring rbcL sequences.

The invention provides shuffled rbcS encoding sequences, wherein the shuffled sequences comprise portions of a first parental rbcS encoding sequence which comprises at least one mutation in the encoding sequence as compared to the collection of predetermined naturally occurring rbcS sequences.

Generally, the nomenclature used hereafter and the laboratory procedures in cell culture, molecular genetics, virology, and nucleic acid chemistry and hybridization described below are those well known and commonly employed in the art. Standard techniques are used for recombinant nucleic acid methods, polynucleotide synthesis, and microbial culture and transformation (e.g., biolistics, Agrobacterium (Ti plasmid), electroporation, lipofection). Generally enzymatic reactions and purification steps are performed according to the manufacturer's specifications. The techniques and procedures are generally performed according to conventional methods in the art and various general references (see, generally, Sambrook et al. Molecular Cloning: A Laboratory Manual, 2d ed. (1989) Cold Spring Harbor Laboratory Press, Cold Spring Harbor, N.Y., which is incorporated herein by reference) which are provided throughout this document. The procedures therein are believed to be well known in the art and are provided for the convenience of the reader. All the information contained therein is incorporated herein by reference.

Oligonucleotides can be synthesized on an Applied Bio Systems oligonucleotide synthesizer according to specifications provided by the manufacturer.

Methods for PCR amplification are described in the art (PCR Technology: Principles and Applications for DNA Amplification ed. H A Erlich, Freeman Press, New York, N.Y. (1992); PCR Protocols: A Guide to Methods and Applications, eds. Innis, Gelfland, Snisky, and White, Academic Press, San Diego, Calif. (1990); Mattila et al. (1991) Nucleic Acids Res. 19: 4967; Eckert, K. A. and Kunkel, T. A. (1991) PCR Methods and Applications 1: 17; PCR, eds. McPherson, Quirkes, and Taylor, IRL Press, Oxford; and U.S. Pat. No. 4,683,202, which are incorporated herein by reference). Leaf PCR is suitable for genotype analysis of transgenote plants.

All sequences referred to herein or equivalents which function in the disclosed methods can be retrieved by GenBank database file designation or a commonly used reference name which is indexed in GenBank or otherwise published are incorporated herein by reference and are publicly available. Over 1,000 Rubisco homologues are available, e.g., in GenBank.

### Incorporation by Reference of Related Applications

The following co-pending patent applications and publications of the present inventors and co-workers are incorporated herein by reference for all purposes: U.S. Ser. No. 08/198,431, filed 17 Feb. 1994, PCT/US95/02126 filed 17 Feb. 1995, WO97/20078, U.S. Pat. No. 5,605,793, U.S. Pat. No. 5,358,665, U.S. Pat. No. 5,270,170, U.S. Ser. No. 08/425,684 filed 18 Apr. 1995, U.S. Ser. No. 08/537,874 filed 30 Oct. 1995, U.S. Ser. No. 08/564,955 filed 30 Nov. 1995, U.S. Ser. No. 08/621,859 filed 25 Mar. 1996, PCT/US96/05480 filed 18 Apr. 1996, U.S. Ser. No. 08/650,400 filed 20 May 1996, U.S. Ser. No. 08/675,502 filed 3 Jul. 1996, U.S. Ser. No. 08/721,824 filed 27 Sep. 1996, U.S. Ser. No. 08/722,660 filed 27 Sep. 1996, and U.S. Ser. No. 08/769,062 filed 18 Dec. 1996; WO98/13485 and WO98/13487; and Stemmer (1995) Science 270: 1510; Stemmer et al. (1995) Gene 164: 49-53; Stemmer (1995) Bio/Technology 13: 549-553; Stemmer (1994) PNAS 91: 10747-10751; Stemmer (1994) Nature 370: 389-391; Crameri et al. (1996) Nature Medicine 2: 1-3; Crameri et al. (1996) Nature Biotechnology 14: 315-319 and; commonly assigned [U.S. Patent Application U.S. Ser. No. 60/107,757 entitled “MODIFIED PHOSPHOENOLPYRUVATE CARBOXYLASE FOR IMPROVEMENT AND OPTIMIZATION OF PLANT PHENOTYPES” filed on 10 Nov. 1998 (Attorney Docket Number 018097-029100US); commonly assigned U.S. Patent Application U.S. Ser. No. 60/107,782, entitled “MODIFIED ADP-GLUCOSE PYROPHOSPHORYLASE FOR IMPROVEMENT AND OPTIMIZATION OF PLANT PHENOTYPES” filed on 10 Nov. 1998 (Attorney docket number 018097-029000US); and “TRANSFORMATION, SELECTION, AND SCREENING OF SEQUENCE SHUFFLED POLYNUCLEOTIDES FOR DEVELOPMENT AND OPTIMIZATION OF PLANT PHENOTYPES” U.S. Ser. No. 60/098,528, PCT/US99/19732 and U.S. Ser. No. 09/385,833 filed Aug. 31, 1998, Aug. 30, 1999, and Aug. 30, 1999, respectively.

### Overview

The invention relates in part to a method for generating novel or improved Rubisco genetic sequences and improved carbon fixation phenotypes which do not naturally occur or would be anticipated to occur at a substantial frequency in nature. A broad aspect of the method employs recursive nucleotide sequence recombination, termed “sequence shuffling” which enables the rapid generation of a collection of broadly diverse phenotypes that can be selectively bred for a broader range of novel phenotypes or more extreme phenotypes than would otherwise occur by natural evolution in the same time period. A basic variation of the method is a recursive process comprising: (1) sequence shuffling of a plurality of species of a genetic sequence, which species may differ by as little as a single nucleotide difference or may be substantially different yet retain sufficient regions of sequence similarity or site-specific recombination junction sites to support shuffling recombination, (2) selection of the resultant shuffled genetic sequence to isolate or enrich a plurality of shuffled genetic sequences having a desired phenotype(s), and (3) repeating steps (1) and (2) on the plurality of shuffled genetic sequences having the desired phenotype(s) until one or more variant genetic sequences encoding a sufficiently optimized desired phenotype is obtained. In this general manner, the method facilitates the “forced evolution” of a novel or improved genetic sequence to encode a desired Rubisco enzymatic phenotype which natural selection and evolution has heretofore not generated in the reference agricultural organism.

Typically, a plurality of Rubisco genetic sequences are shuffled and selected by the present method. The method can be used with a plurality of alleles, homologs, or cognate genes of a gentic locus, or even with a plurality or genetic sequences from related organisms, and in some instances with unrelated genetic sequences or portions thereof which have recombinogenic portions (either naturally or generated via genetic engineering). Furthermore, the method can be used to evolve a heterologous Rubisco sequence (e.g., a non-naturally occurring mutant gene, or a subunit from another species) to optimize its function in concert with a complementing subunit, and/or in a particular host cell.

### Rubisco

An example of such a biosynthetic pathway enzyme is ribulose-1,5-bisphosphate carboxylase-oxygenase (“Rubisco”), which is the enzyme in plants, green algae (including marine algae), and photosynthetic bacteria involved in fixing atmospheric carbon dioxide into reduced sugars. Rubisco is a true bifunctional enzyme; it catalyzes (i) carboxylation of ribulose bisphosphate (“RuBP”) to form two molecules of 3-phosphoglycerate, and (ii) oxygenation of rubp to form one molecule of 3-phosphoglycerate and one molecule of 2-phosphoglycerate, at the same active site. The oxygenation reaction catalyzed by Rubisco (also called photorespiration) is a “wasteful” process, since it significantly reduces the amount of carbon fixed. Both CO₂ and O₂ compete for the same active site, although the Km for CO₂ is about an order of magnitude less than for O₂. In plants, as the temperature rises during the course of the day, photorespiration catalyzed by Rubisco increases relative to carbon fixation, reducing the energy efficiency of carbon fixation. This is because the solubility of CO₂ decreases with increasing temperature relative to O₂. During the course of evolution, Rubisco has been selected for carboxylation specificity (carboxylation specificity factor defined as the ratio of velocity of carboxylation×Km for O₂ to velocity of oxygenation×Km for CO₂). This specificity has evolved from about 10 in bacteria, to 50 in cyanobacteria, and to about 80 in higher plants. In photosynthetic bacteria and dinoflagelates, Rubisco is present as a dimer of a large subunit (Form II, L₂), and no small subunit is present. In cyanobacteria, green algae, and higher plants (C3 and C4 plants), Rubisco is present as multimeric (e.g., hexadecimeric) protein composed of two subunits, the large (L) subunit which is catalytic, and the small (S) subunit which is regulatory, formed into an enzymatically active multimer (e.g., L₈S₈ hexadecimer). Coding sequences for L and S subunits for various species are disclosed in the literature and Genbank, among other public sources, and may be obtained by cloning, PCR, or from deposited materials.

Rubisco subunit shufflants are generated by any suitable shuffling method as noted above from one or more parental sequences, optionally including mutagenesis, in vitro manipulation, in vivo manipulation of sequences or in silico manipulation of sequences, and the resultant shufflants are introduced into a suitable host cell, typically in the form of expression cassettes wherein the shuffled polynucleotide sequence encoding the Rubisco subunit is operably linked to a transcriptional regulatory sequence and any necessary sequences for ensuring transcription, translation, and processing of the encoded Runbisco subunit protein. Each such expression cassette or its shuffled Rubisco encoding sequence can be referred to as a “library member” composing a library of shuffled Rubisco subunit sequences. The library is introduced into a population of host cells, such that individual host cells receive substantially one or a few species of library member(s), to form a population of shufflant host cells expressing a library of shuffled Rubisco subunit species. The population of shufflant host cells is screened so as to isolate or segregate host cells and/or their progeny which express Rubisco subunit(s) having the desired enhanced phenotype. The shuffled Rubisco subunit encoding sequence(s) is/are recovered from the isolated or segregated shufflant host cells, and typically subjected to at least one subsequent round of mutagenesis and/or sequence shuffling, introduced into suitable host cells, and selected for the desired enhanced enzymatic phenotype; this cycle is generally performed iteratively until the shufflant host cells express a Rubisco subunit having the desired level or enzymatic phenotype or until the rate of improvement in the desired enzymatic phenotype produced by shuffling has substantially plateaued. The shufflant Rubisco polynucleotides expressed in the host cells following the iterative process of shuffling and selection encode Rubisco subunit specie(s) having the desired enhanced phenotype.

For illustration and not to limit the invention, examples of a desired Rubisco enzymatic phenotype can include increased RuBP carboxylase rate, decreased RuBP oxygenase rate, increased Km for O₂, decreased Km for CO₂, decreased ratio of Km for CO₂ to Km for O₂, velocity for O₂ or CO₂, and the like as described herein and as may be desired by the skilled artisan.

A variety of Rubisco gene and gene homologue sources are known and can be used in the recombination processes herein. For example, as noted, a variety of references herein describe such genes. For example, Croy, (ed.) (1993) Plant Molecular Biology Bios Scientific Publishers, Oxford, U.K. describe several Rubisco genes and sequence sources in public databases. Examples of public databases that include Rubisco sources include: Genbank: www.ncbi.nlm.nih.gov/genbank/; EMBL: www.ebi.ac.uk.embl/; as well as, e.g., the protein databank, Brookhaven Laboratories; the University of Wisconsin Biothechology Center, the DNA databank of Japan, Laboratory of genetic Information Research, Misuina, Shizuda, Japan. As noted, over 1,000 different Rubisco homologues are available in Genbank alone. In addition, specific internet sites which provide information regarding Rubisco include, e.g., http://ss.tnaes.affrc.go.jp/pub/suzuki/rubisco.html; http://icdweb.cc.purdue.edu/˜knollje/Rubisco.html; http://www.agron.missouri.edu/cgi-bin/sybgw_mdb/mdb3/Locus/114858; http://gdb.wehi.edu.au/scop/data/scop.1.004.037.001.000.000.html; http://www.blc.arizona.edu/courses/181gh/rick/photosynthesis/Calvin.html; http://www.tarweed.com/pgr/PGR98-207.html; and http://homepage.ruhr-uni-bochum.de/Marc.Saric/rubisco3.html.

Shuffling

The following publications describe a variety of recursive recombination procedures and/or methods which can be incorporated into such procedures, e.g., for shuffling of Rubisco genes and gene fragments as herein: Stemmer, et al., (1999) “Molecular breeding of viruses for targeting and other clinical properties. Tumor Targeting” 4:1-4; Nesset al. (1999) “DNA Shuffling of subgenomic sequences of subtilisin” Nature Biotechnology 17:893-896; Chang et al. (1999) “Evolution of a cytokine using DNA family shuffling” Nature Biotechnology 17:793-797; Minshull and Stemmer (1999) “Protein evolution by molecular breeding” Current Opinion in Chemical Biology 3:284-290; Christians et al. (1999) “Directed evolution of thymidine kinase for AZT phosphorylation using DNA family shuffling” Nature Biotechnology 17:259-264; Crameriet al. (1998) “DNA shuffling of a family of genes from diverse species accelerates directed evolution” Nature 391:288-291; Crameri et al. (1997) “Molecular evolution of an arsenate detoxification pathway by DNA shuffling,” Nature Biotechnology 15:436-438; Zhang et al. (1997) “Directed evolution of an effective fucosidase from a galactosidase by DNA shuffling and screening” Proceedings of the National Academy of Sciences, U.S.A. 94:4504-4509; Patten et al. (1997) “Applications of DNA Shuffling to Pharmaceuticals and Vaccines” Current Opinion in Biotechnology 8:724-733; Crameri et al. (1996) “Construction and evolution of antibody-phage libraries by DNA shuffling” Nature Medicine 2:100-103; Crameri et al. (1996) “Improved green fluorescent protein by molecular evolution using DNA shuffling” Nature Biotechnology 14:315-319; Gates et al. (1996) “Affinity selective isolation of ligands from peptide libraries through display on a lac repressor ‘headpiece dimer’” Journal of Molecular Biology 255:373-386; Stemmer (1996) “Sexual PCR and Assembly PCR” In: The Encyclopedia of Molecular Biology. VCH Publishers, New York. pp. 447-457; Crameri and Stemmer (1995) “Combinatorial multiple cassette mutagenesis creates all the permutations of mutant and wildtype cassettes” BioTechniques 18:194-195; Stemmer et al., (1995) “Single-step assembly of a gene and entire plasmid form large numbers of oligodeoxyribonucleotides” Gene, 164:49-53; Stemmer (1995) “The Evolution of Molecular Computation” Science 270: 1510; Stemmer (1995) “Searching Sequence Space” Bio/Technology 13:549-553; Stemmer (1994) “Rapid evolution of a protein in vitro by DNA shuffling” Nature 370:389-391; and Stemmer (1994) “DNA shuffling by random fragmentation and reassembly: In vitro recombination for molecular evolution.” Proceedings of the National Academy of Sciences, U.S.A. 91:10747-10751.

Additional details regarding DNA shuffling methods are found in U.S. Patents by the inventors and their co-workers, including: U.S. Pat. No. 5,605,793 to Stemmer (Feb. 25, 1997), “METHODS FOR IN VITRO RECOMBINATION;” U.S. Pat. No. 5,811,238 to Stemmer et al. (Sep. 22, 1998) “METHODS FOR GENERATING POLYNUCLEOTIDES HAVING DESIRED CHARACTERISTICS BY ITERATIVE SELECTION AND RECOMBINATION;” U.S. Pat. No. 5,830,721 to Stemmer et al. (Nov. 3, 1998), “DNA MUTAGENESIS BY RANDOM FRAGMENTATION AND REASSEMBLY;” U.S. Pat. No. 5,834,252 to Stemmer, et al. (Nov. 10, 1998) “END-COMPLEMENTARY POLYMERASE REACTION,” and U.S. Pat. No. 5,837,458 to Minshull, et al. (Nov. 17, 1998), “METHODS AND COMPOSITIONS FOR CELLULAR AND METABOLIC ENGINEERING.”

In addition, details and formats for DNA shuffling are found in a variety of PCT and foreign patent application publications, including: Stemmer and Crameri, “DNA MUTAGENESIS BY RANDOM FRAGMENTATION AND REASEMBLY” WO 95/22625; Stemmer and Lipschutz “END COMPLEMENTARY POLYMERASE CHAIN REACTION” WO 96/33207; Stemmer and Crameri “METHODS FOR GENERATING POLYNUCLEOTIDES HAVING DESIRED CHARACTERISTICS BY ITERATIVE SELECTION AND RECOMBINATION” WO 97/0078; Minshul and Stemmer, “METHODS AND COMPOSITIONS FOR CELLULAR AND METABOLIC ENGINEERING” WO 97/35966; Punnonen et al. “TARGETING OF GENETIC VACCINE VECTORS” WO 99/41402; Punnonen et al. “ANTIGEN LIBRARY IMMUNIZATION” WO 99/41383; Punnonen et al. “GENETIC VACCINE VECTOR ENGINEERING” WO 99/41369; Punnonen et al. OPTIMIZATION OF IMMUNOMODULATORY PROPERTIES OF GENETIC VACCINES WO 9941368; Stemmer and Crameri, “DNA MUTAGENESIS BY RANDOM FRAGMENTATION AND REASSEMBLY” EP 0934999; Stemmer “EVOLVING CELLULAR DNA UPTAKE BY RECURSIVE SEQUENCE RECOMBINATION” EP 0932670; Stemmer et al., “MODIFICATION OF VIRUS TROPISM AND HOST RANGE BY VIRAL GENOME SHUFFLING” WO 9923107; Apt et al., “HUMAN PAPILLOMAVIRUS VECTORS” WO 9921979; Del Cardayre et al. “EVOLUTION OF WHOLE CELLS AND ORGANISMS BY RECURSIVE SEQUENCE RECOMBINATION” WO 9831837; Patten and Stemmer, “METHODS AND COMPOSITIONS FOR POLYPEPTIDE ENGINEERING” WO 9827230; Stemmer et al., and “METHODS FOR OPTIMIZATION OF GENE “THERAPY BY RECURSIVE SEQUENCE SHUFFLING AND SELECTION” WO9813487.

Certain U.S. Applications provide additional details regarding DNA shuffling and related techniques, including “SHUFFLING OF CODON ALTERED GENES” by Patten et al. filed Sep. 29, 1998, (U.S. Ser. No. 60/102,362), Jan. 29, 1999 (U.S. Ser. No. 60/117,729), and Sep. 28, 1999, U.S. Ser. No. 09/407,800 (Attorney Docket Number 20-28520US/PCT); “EVOLUTION OF WHOLE CELLS AND ORGANISMS BY RECURSIVE SEQUENCE RECOMBINATION”, by del Cardyre et al. filed Jul. 15, 1998 (U.S. Ser. No. 09/166,188), and Jul. 15, 1999 (U.S. Ser. No. 09/354,922); “OLIGONUCLEOTIDE MEDIATED NUCLEIC ACID RECOMBINATION” by Crameri et al., filed Feb. 5, 1999 (U.S. Ser. No. 60/118,813) and filed Jun. 24, 1999 (U.S. Ser. No. 60/141,049) and filed Sep. 28, 1999 (U.S. Ser. No. 09/408,392, Attorney Docket Number 02-29620US); and “USE OF CODON-BASED OLIGONUCLEOTIDE SYNTHESIS FOR SYNTHETIC SHUFFLING” by Welch et al., filed Sep. 28, 1999 (U.S. Ser. No. 09/408,393, Attorney Docket Number 02-010070US); and “METHODS FOR MAKING CHARACTER STRINGS, POLYNUCLEOTIDES & POLYPEPTIDES HAVING DESIRED CHARACTERISTICS” by Selifonov and Stemmer, filed Feb. 5, 1999 (U.S. Ser. No. 60/118,854) and “METHODS FOR MAKING CHARACTER STRINGS, POLYNUCLEOTIDES & POLYPEPTIDES HAVING DESIRED CHARACTERISTICS” by Selifonov et al. filed Oct. 12, 1999 (U.S. Ser. No. 09/416,375).

As review of the foregoing publications, patents, published applications and U.S. patent applications reveals, recursive recombination and selection of nucleic acids to provide new nucleic acids with desired properties can be carried out by a number of established methods. Any of these methods can be adapted to the present invention to evolve Rubisco coding nucleic acids or homolgues to produce new enzymes with improved properties. Both the methods of making such enzymes and the enzymes or enzyme coding libraries produced by these methods are a feature of the invention.

In brief, at least 5 different general classes of recombination methods are applicable to the present invention. First, nucleic acids can be recombined in vitro by any of a variety of techniques discussed in the references above, including e.g., DNAse digestion of nucleic acids to be recombined followed by ligation and/or PCR reassembly of the nucleic acids. Second, nucleic acids can be recursively recombined in vivo, e.g., by allowing recombination to occur between nucleic acids in cells. Third, whole cell genome recombination methods can be used in which whole genomes of cells are recombined, optionally including spiking of the genomic or chloroplast recombination mixtures with desired library components such as Rubisco encoding nucleic acids. Fourth, synthetic recombination methods can be used, in which oligonucleotides corresponding to different Rubisco homologues are synthesized and reassembled in PCR or ligation reactions which include oligonucleotides which correspond to more than one parental nucleic acid, thereby generating new recombined nucleic acids. Oligonucleotides can be made by standard nucleotide addition methods, or can be made, e.g., by tri-nucleotide synthetic approaches. Fifth, in silico methods of recombination can be effected in which genetic algorithms are used in a computer to recombine sequence strings which correspond to Rubisco homologues. The resulting recombined sequence strings are optionally converted into nucleic acids by synthesis of nucleic acids which correspond to the recombined sequences, e.g., in concert with oligonucleotide synthesis/gene reassembly techniques. Any of the preceding general recombination formats can be practiced in a reiterative fashion to generate a more diverse set of recombinant nucleic acids.

The above references provide these and other basic recombination formats as well as many modifications of these formats. Regardless of the format which is used, the nucleic acids of the invention can be recombined (with each other or with related (or even unrelated) nucleic acids to produce a diverse set of recombinant nucleic acids, including homologous nucleic acids.

Following recombination, any nucleic acids which are produced can be selected for a desired activity. A variety of related (or even unrelated) properties can be assayed for, using any available assay.

One basic format of shuffling consists of a method for generating a selected polynucleotide sequence or population of selected polynucleotide sequences, typically in the form of amplified and/or cloned polynucleotides, whereby the selected polynucleotide sequence(s) possess or encode a desired phenotypic characteristic (e.g., encode a polypeptide, promote transcription of linked polynucleotides, modify transformation efficiency, bind a protein, and the like) which can be selected for. One method of identifying polypeptides that possess a desired structural or functional property, such as encoding a desired enzymatic function(s) (e.g., an enhanced Rubisco, a herbicide catabolizing enzyme, an optimized plant biosynthetic pathway), involves the screening of a large library of polynucleotides for individual library members which possess or encode the desired structure or functional property conferred by the polynucleotide sequence.

In a general aspect, the invention provides a sequence shuffling method, for generating libraries of recombinant polynucleotides having a desired Rubisco enzyme characteristic which can be selected or screened for. Libraries of recombinant polynucleotides are generated from a population of related-sequence polynucleotides which comprise sequence regions which have substantial sequence identity and can be homologously recombined in vitro or in vivo. In the method, at least two species of the related-sequence polynucleotides are combined in a recombination system suitable for generating sequence-recombined polynucleotides, wherein said sequence-recombined polynucleotides comprise a portion of at least one first species of a related-sequence polynucleotide with at least one adjacent portion of at least one second species of a related-sequence polynucleotide. Recombination systems suitable for generating sequence-recombined polynucleotides can be either: (1) in vitro systems for homologous recombination or sequence shuffling via amplification or other formats described herein, or (2) in vivo systems for homologous recombination or site-specific recombination as described herein.

The population of sequence-recombined polynucleotides comprises a subpopulation of polynucleotides which possess desired or advantageous characteristics and which can be selected by a suitable selection or screening method. The selected sequence-recombined polynucleotides, which are typically related-sequence polynucleotides, can then be subjected to at least one recursive cycle wherein at least one selected sequence-recombined polynucleotide is combined with at least one distinct species of related-sequence polynucleotide (which may itself be a selected sequence-recombined polynucleotide) in a recombination system suitable for generating sequence-recombined polynucleotides, such that additional generations of sequence-recombined polynucleotide sequences are generated from the selected sequence-recombined polynucleotides obtained by the selection or screening method employed. In this manner, recursive sequence recombination generates library members which are sequence-recombined polynucleotides possessing desired characteristics. Such characteristics can be any property or attribute capable of being selected for or detected in a screening system, and may include properties of: an encoded protein, a transcriptional element, a sequence controlling transcription, RNA processing, RNA stability, chromatin conformation, translation, or other expression property of a gene or transgene, a replicative element, a protein-binding element, or the like, such as any feature which confers a selectable or detectable property.

Nucleic acid sequence shuffling is a method for recursive in vitro or in vivo homologous or nonhomologous recombination of pools of nucleic acid fragments or polynucleotides (e.g., genes from agricultural organisms or portions thereof). Mixtures of related nucleic acid sequences or polynucleotides are randomly or pseudorandomly fragmented, and reassembled to yield a library or mixed population of recombinant nucleic acid molecules or polynucleotides.

The present invention is directed to a method for generating a selected polynucleotide sequence (e.g., a plant rbc gene or microbe rbc gene, or combinations thereof) or population of selected polynucleotide sequences, typically in the form of amplified and/or cloned polynucleotides, whereby the selected polynucleotide sequence(s) possess a desired phenotypic characteristic of Rubisco enzymes or subunits thereof which can be selected for, and whereby the selected polynucleotide sequences are genetic sequences having a desired functionality and/or conferring a desired phenotypic property to an agricultural organism in which the polynucleotide has been transferred into.

In a general aspect, the invention provides a method, called “sequence shuffling,” for generating libraries of recombinant polynucleotides having a subpopopulation of library members which encode an enhanced or improved Rubisco L or S protein. Libraries of recombinant polynucleotides are generated from a population of related-sequence Rubisco polynucleotides which comprise sequence regions which have substantial sequence identity and can be homologously recombined in vitro or in vivo. In the method, at least two species of the related-sequence Rubisco polynucleotides are combined in a recombination system suitable for generating sequence-recombined polynucleotides, wherein said sequence-recombined polynucleotides comprise a portion of at least one first species of a related-sequence Rubisco polynucleotide with at least one adjacent portion of at least one second species of a related-sequence Rubisco polynucleotide. Recombination systems suitable for generating sequence-recombined polynucleotides can be either: (1) in vitro systems for homologous recombination or sequence shuffling via amplification or other formats described herein, or (2) in vivo systems for homologous recombination or site-specific recombination as described herein, or template-switching of a retroviral genome replication event. The population of sequence-recombined polynucleotides comprises a subpopulation of Rubisco polynucleotides which possess desired or advantageous enzymatic characteristics and which can be selected by a suitable selection or screening method. The selected sequence-recombined Rubisco polynucleotides, which are typically related-sequence polynucleotides, can then be subjected to at least one recursive cycle wherein at least one selected sequence-recombined Rubisco polynucleotide is combined with at least one distinct species of related-sequence Rubisco polynucleotide (which may itself be a selected sequence-recombined polynucleotide) in a recombination system suitable for generating sequence-recombined Rubisco polynucleotides, such that additional generations of sequence-recombined polynucleotide sequences are generated from the selected sequence-recombined polynucleotides obtained by the selection or screening method employed. In this manner, recursive sequence recombination generates library members which are sequence-recombined polynucleotides possessing desired Rubisco enzymatic characteristics. Such characteristics can be any property or attribute capable of being selected for or detected in a screening system.

Screening/selection produces a subpopulation of genetic sequences (or cells) expressing recombinant forms of Rubisco subunit gene(s) that have evolved toward acquisition of a desired enzymatic property. These recombinant forms can then be subjected to further rounds of recombination and screening/selection in any order. For example, a second round of screening/selection can be performed analogous to the first resulting in greater enrichment for genes having evolved toward acquisition of the desired enzymatic property. Optionally, the stringency of selection can be increased between rounds (e.g., if selecting for drug resistance, the concentration of drug in the media can be increased). Further rounds of recombination can also be performed by an analogous strategy to the first round generating further recombinant forms of the gene(s) or genome(s). Alternatively, further rounds of recombination can be performed by any of the other molecular breeding formats discussed. Eventually, a recombinant form of the Rubisco subunit gene(s) is generated that has fully acquired the desired enzymatic property.

In an embodiment, the first plurality of selected library members is fragmented and homologously recombined by PCR in vitro. Fragment generation is by nuclease digestion, partial extension PCR amplification, PCR stuttering, or other suitable fragmenting means, such as described herein and in WO95/22625 published 24 Aug. 1995, and in commonly owned U.S. Ser. No. 08/621,859 filed 25 Mar. 1996, PCT/US96/05480 filed 18 Apr. 1996, which are incorporated herein by reference). Stuttering is fragmentation by incomplete polymerase extension of templates. A recombination format based on very short PCR extension times can be employed to create partial PCR products, which continue to extend off a different template in the next (and subsequent) cycle(s), and effect de facto fragmentation. Template-switching and other formats which accomplish sequence shuffling between a plurality of sequence-related polynucleotides can be used. Such alternative formats will be apparent to those skilled in the art.

In an embodiment, the first plurality of selected library members is fragmented in vitro, the resultant fragments transferred into a host cell or organism and homologously recombined to form shuffled library members in vivo.

In an embodiment, the first plurality of selected library members is cloned or amplified on episomally replicable vectors, a multiplicity of said vectors is transferred into a cell and homologously recombined to form shuffled library members in vivo.

In an embodiment, the first plurality of selected library members is not fragmented, but is cloned or amplified on an episomally replicable vector as a direct repeat or indirect (or inverted) repeat, which each repeat comprising a distinct species of selected library member sequence, said vector is transferred into a cell and homologously recombined by intra-vector or inter-vector recombination to form shuffled library members in vivo.

In an embodiment, combinations of in vitro and in vivo shuffling are provided to enhance combinatorial diversity. The recombination cycles (in vitro or in vivo) can be performed in any order desired by the practitioner.

In one embodiment, the first plurality of selected library members is fragmented and homologously recombined by PCR in vitro. Fragment generation is by nuclease digestion, partial extension PCR amplification, PCR stuttering, or other suitable fragmenting means, such as described herein and in the documents incorproated herein by reference. Stuttering is fragmentation by incomplete polymerase extension of templates.

In one embodiment, the first plurality of selected library members is fragmented in vitro, the resultant fragments transferred into a host cell or organism and homologously recombined to form shuffled library members in vivo. In an aspect, the host cell is a plant cell which has been engineered to contain enhanced recombination systems, such as an enhanced system for general homologous recombination (e.g., a plant expressing a recA protein or a plant recombinase from a transgene or plant virus) or a site-specific recombination system (e.g., a cre/LOX or frt/FLP system encoded on a transgene or plant virus).

In one embodiment, the first plurality of selected library members is cloned or amplified on episomally replicable vectors, a multiplicity of said vectors is transferred into a cell and homologously recombined to form shuffled library members in vivo in a plant cell, algae cell, or bacterial cell. Other cell types may be used, if desired.

In one embodiment, the first plurality of selected library members is not fragmented, but is cloned or amplified on an episomally replicable vector as a direct repeat or indirect (or inverted) repeat, which each repeat comprising a distinct species of selected library member sequence, said vector is transferred into a cell and homologously recombined by intra-vector or inter-vector recombination to form shuffled library members in vivo in a plant cell, algae cell, or microorganism.

In an embodiment, the method employs at least one parental polynucleotide sequence that encodes a Rubisco subunit of a marine algae, such as for example and not limitation Cylindrotheca fusiformis, Olisthodiscus luteus, Cryptomonas, and Porphyridium, among others having Rubisco enzymes with a high ratio of carboxylase to oxygenase activity (Read B A and Tabita F R (1994) Arch. Biochem. Biophys. 312:210).

In an embodiment, combinations of in vitro and in vivo shuffling are provided to enhance combinatorial diversity.

At least two additional related specific formats are useful in the practice of the present invention. The first, referred to as “in silico” shuffling utilizes computer algorithms to perform “virtual” shuffling using genetic operators in a computer. As applied to the present invention, Calvin or Krebs cycle enzymes such as Rubisco nucleic acid sequence strings are recombined in a computer system and desirable products are made, e.g., by reassembly PCR or ligation of synthetic oligonucleotides, or other available techniques. In silico shuffling is described in detail in Selifonov and Stemmer in “METHODS FOR MAKING CHARACTER STRINGS, POLYNUCLEOTIDES & POLYPEPTIDES HAVING DESIRED CHARACTERISTICS” filed Feb. 5, 1999, U.S. Ser. No. 60/118,854 and “METHODS FOR MAKING CHARACTER STRINGS, POLYNUCLEOTIDES & POLYPEPTIDES HAVING DESIRED CHARACTERISTICS” by Selifonov et al. filed Oct. 12, 1999 (U.S. Ser. No. 09/416,375). In brief, genetic operators (algorithms which represent given genetic events such as point mutations, recombination of two strands of homologous nucleic acids, etc.) are used to model recombinational or mutational events which can occur in one or more nucleic acid, e.g., by aligning nucleic acid sequence strings (using standard alignment software, or by manual inspection and alignment) and predicting recombinational outcomes based upon selected genetic algorithms (mutation, recombination, etc.). The predicted recombinational outcomes are used to produce corresponding molecules, e.g., by oligonucleotide synthesis and reassembly PCR. As applied to the present invention, Rubisco and other Calvin or Krebs cycle nucleic acids are aligned and recombined in silico, using any desired genetic operator, to produce character strings which are then generated synthetically for subsequent screening.

The second useful format is referred to as “oligonucleotide mediated shuffling” in which oligonucleotides corresponding to a family of related homologous nucleic acids (e.g., as applied to the present invention, families of homologous Rubisco variants of a nucleic acid) which are recombined to produce selectable nucleic acids. This format is described in detail in Crameri et al. “OLIGONUCLEOTIDE MEDIATED NUCLEIC ACID RECOMBINATION” filed Feb. 5, 1999, U.S. Ser. No. 60/118,813, Crameri et al. “OLIGONUCLEOTIDE MEDIATED NUCLEIC ACID RECOMBINATION” filed Jun. 24, 1999, U.S. Ser. No. 60/141,049; Crameri et al. “OLIGONUCLEOTIDE MEDIATED NUCLEIC ACID RECOMBINATION” filed Sep. 28, 1999 (U.S. Ser. No. 09/408,392, Attorney Docket Number 02-29620US); and “USE OF CODON-BASED OLIGONUCLEOTIDE SYNTHESIS FOR SYNTHETIC SHUFFLING” by Welch et al., filed Sep. 28, 1999 (U.S. Ser. No. 09/408,393, Attorney Docket Number 02-010070US). In brief, selected oligonucleotides corresponding to multiple homologous parental nucleic acids are synthesized, ligated and elongated (typically in a recursive format), typically either in a polymerase or ligase-mediated elongation reaction, to produce full-length Rubisco nucleic acids. The technique can be used to recombine homologous or even non-homologous Rubisco nucleic acid sequences.

One advantage of oligonucleotide-mediated recombination is the ability to recombine homologous nucleic acids with low sequence similarity, or even non-homologous nucleic acids. In these low-homology oligonucleotide shuffling methods, one or more set of fragmented nucleic acids (e.g., oligonucleotides corresponding to multiple Rubisco nucleic acids) are recombined, e.g., with a set of crossover family diversity oligonucleotides. Each of these crossover oligonucleotides have a plurality of sequence diversity domains corresponding to a plurality of sequence diversity domains from homologous or non-homologous nucleic acids with low sequence similarity. The fragmented oligonucleotides, which are derived by comparison to one or more homologous or non-homologous nucleic acids, can hybridize to one or more region of the crossover oligos, facilitating recombination.

When recombining homologous nucleic acids, sets of overlapping family gene shuffling oligonucleotides (which are derived by comparison of homologous nucleic acids, by synthesis of corresponding oligonucleotides) are hybridized and elongated (e.g., by reassembly PCR or ligation), providing a population of recombined nucleic acids, which can be selected for a desired trait or property. The set of overlapping family shuffling gene oligonucleotides includes a plurality of oligonucleotide member types which have consensus region subsequences derived from a plurality of homologous target nucleic acids.

Typically, as applied to the present invention, family gene shuffling oligonucleotides which include one or more Rubisco nucleic acid(s) are provided by aligning homologous nucleic acid sequences to select conserved regions of sequence identity and regions of sequence diversity. A plurality of family gene shuffling oligonucleotides are synthesized (serially or in parallel) which correspond to at least one region of sequence diversity.

Sets of fragments, or subsets of fragments used in oligonucleotide shuffling approaches can be provided by cleaving one or more homologous nucleic acids (e.g., with a DNase), or, more commonly, by synthesizing a set of oligonucleotides corresponding to a plurality of regions of at least one nucleic acid (typically oligonucleotides corresponding to a full-length nucleic acid are provided as members of a set of nucleic acid fragments). In the shuffling procedures herein, these cleavage fragments can be used in conjunction with family gene shuffling oligonucleotides, e.g., in one or more recombination reaction to produce recombinant Rubisco nucleic acid(s).

One final synthetic variant worth noting is found in “SHUFFLING OF CODON ALTERED GENES” by Patten et al. filed Sep. 29, 1998, (U.S. Ser. No. 60/102,362), Jan. 29, 1999 (U.S. Ser. No. 60/117,729), and Sep. 28, 1999, PCT/US99/22588 (Attorney Docket Number 20-28520US/PCT). As noted in detail in this set of related applications, one way of generating diversity in a set of nucleic acids to be shuffled (i.e., as applied to the present invention, Rubisco nucleic acids), is to provide codon-altered nucleic acids which can be shuffled to provide access to sequence space not present in naturally occurring sequences. In brief, by synthesizing nucleic acids in which the codons which encode polypeptides are altered, it is possible to access a completely different mutational spectrum upon subsequent mutation of the nucleic acid. This increases the sequence diversity of the starting nucleic acids for shuffling protocols, which alters the rate and results of forced evolution procedures. Codon modification procedures can be used to modify any Rubisco nucleic acid or shuffled nucleic acid, e.g., prior to performing DNA shuffling.

In brief, oligonucleotide sets comprising codon variations are synthesized and reassembled into full-length nucleic acids. The full length nucleic acids can themselves be shuffled (e.g., where the oligonucleotides to be reassembled provide sequence diversity at selected sites), and/or the full-length sequences can be shuffled by any available procedure to produce diverse sets of Rubisco nucleic acids.

Improved Plants

Without reciting the various generalized formats of polynucleotide sequence shuffling and selection described previously or herein below, which will be referred to herein by the shorthand “shuffling”, the present invention provides methods, compositions, and uses related to creating novel or improved plants, plant cells, algal cells, soil microbes, plant pathogens, commensal microbes, or other plant-related organisms having art-recognized importance to the agricultural, horticultural, and argonomic areas (collectively, “agricultural organisms”). In particular, any plant, plant cell, algal cell, etc. can be transduced with a shuffled nucleic acid produced according to the present invention. For example, agronomically and horticulturally important plant species can be transduced. Such species include, but are not restricted to, members of the families: Graminae (including corn, rye, triticale, barley, millet, rice, wheat, oats, etc.); Leguminosae (including pea, beans, lentil, peanut, yam bean, cowpeas, velvet beans, soybean, clover, alfalfa, lupine, vetch, lotus, sweet clover, wisteria, and sweetpea); Compositae (the largest family of vascular plants, including at least 1,000 genera, including important commercial crops such as sunflower) and Rosaciae (including raspberry, apricot, almond, peach, rose, etc.), as well as nut plants (including, walnut, pecan, hazelnut, etc.) Targets for modification the evolved vectors of the invention, as well as those specified above, include plants from the genera: Agrostis, Allium, Anitirrhinum, Apium, Arachis, Asparagus, Atropa, Avena (e.g., oats), Bambusa, Brassica, Bromus, Browaalia, Camellia, Cannabis, Capsicum, Cicer, Chenopodium, Chichorium, Citrus, Coffea, Coix, Cucumis, Curcubita, Cynodon, Dactylis, Datura, Daucus, Digitalis, Dioscorea, Elaeis, Eleusine, Festuca, Fragaria, Geranium, Glycine, Helianthus, Heterocallis, Hevea, Hordeum (e.g., barley), Hyoscyamus, Ipomoea, Lactuca, Lens, Lilium, Linum, Lolium, Lotus, Lycopersicon, Majorana, Malus, Mangifera, Manihot, Medicago, Nemesia, Nicotiana, Onobrychis, Oryza (e.g., rice), Panicum, Pelargonium, Pennisetum (e.g., millet), Petunia, Pisum, Phaseolus, Phleum, Poa, Prunus, Ranunculus, Raphanus, Ribes, Ricinus, Rubus, Saccharum, Salpiglossis, Secale (e.g., rye), Senecio, Setaria, Sinapis, Solanum, Sorghum, Stenotaphrum, Theobroma, Trifolium, Trigonelia, Triticum (e.g., wheat), Vicia, Vigna, Vitis, Zea (e.g., corn), the Olyreae, the Pharoideae and many others.

For example, common crop plants which are targets of the present invention include corn, rice, triticale, rye, cotton, soybean, sorghum, wheat, oats, barley, millet, sunflower, canola, peas, beans, lentils, peanuts, yam beans, cowpeas, velvet beans, clover, alfalfa, lupine, vetch, lotus, sweet clover, wisteria, sweetpea and nut plants (e.g., walnut, pecan, etc).

In certain variations, naturally occurring in vivo recombination mechanisms of plants, agricultural microorganisms, or vector-host cells for intermediate replication can be used in conjunction with a collection of shuffled polynucleotide sequence variants having a desired phenotypic property to be optimized further; in this way, a natural recombination mechanism can be combined with intelligent selection of variants in an iterative manner to produce optimized variants by “forced evolution”, wherein the forced evolved variants are not expected to, nor are observed to, occur in nature, nor are predicted to occur at an appreciable frequency. The practitioner may further elect to supplement and/or the mutational drift by introducing intentionally mutated polynucleotide species suitable for shuffling, or portions thereof, into the pool of initial polynucleotide species and/or into the plurality of selected, shuffled polynucleotide species which are to be recombined. Mutational drift may also be supplemented by the use of mutagens (e.g., chemical mutagens or mutagenic irradiation), or by employing replication conditions which enhance the mutation rate.

### Forced Evolution of Genes

The invention provides a means to evolve Rubisco (rbcS and/or rbcL)gene variants and/or suitable host cells, as well as providing a model system for evaluating a library of agents to identify candidate agents that could find use as agricultural reagents (e.g., herbicide) for commercial applications. Such agents may exhibit selectivity for inhibition of a naturally occurring Rubisco enzyme and may be substantially less effective at inhibiting a shuffled Rubisco enzyme which has been evolved to be resistant to the agent.

### Rubisco Shuffling Combinations

Although the skilled artisan may select alternative shuffling strategies for enhancing Rubisco enzyme properties, the following general combinations can be used:

I. Shuffling a Form II L subunit from a first species of photosynthetic bacteria with a Form II subunit from a second species of photosynthetic bacteria. The resultant shufflants may be transformed into bacterial host cells which preferably lack endogenous Rubisco activity (e.g., E. coli), algal cells, or plant cells for expression and selection. Phenotype selection of shufflants is typically performed by biochemical assay for RuBP carboxylase and/or RuBP oxygenase activity, such as according to Jordan D B and Ogren W L (1981) Nature 291: 513; or other suitable assay method selected by the artisan. Example photosynthetic bacteria for obtaining the rbcL gene(s) include Rhodobacter shaeroides (Falcone et al. (1988) J. Bact. 170: 5), Rhodospirrilum rubrum (Falcone et al. (1991) J. Bact. 173: 2099; Falcone D L and Tabita R (1993) J. Bact. 175: 5066; Narange et al. (1984) Mol. Gen. Genet. 193: 220) ) and the like. A preferred host cell is a strain of photosynthetic bacterium that is transformable (Fitzmaurice et al (1991) Roberts E P (1991) Arch. Microb. 156: 142) and which can be complemented to photoheterotrophic growth by expression of a functional rbcL gene (e.g., cbbM mutant Rubisco deletion strain; I-19 strain).

II. Shuffling a Form II L subunit from a species of photosynthetic bacteria with a Form I subunit from a photosynthetic dinoflagellate. The resultant shufflants may be transformed into bacterial host cells which preferably lack endogenous Rubisco activity (e.g., E. coli), algal cells, or plant cells for expression and selection. Phenotype selection of shufflants is typically performed by biochemical assay for RuBP carboxylase and/or RuBP oxygenase activity, such as according to Jordan D B and Ogren W L (1981) op.cit or other suitable assay method selected by the artisan. Example photosynthetic bacterial sources for the rbcL gene(s) include those from Rhodobacter shaeroides, Rhodospirrilum rubrum and the like. Example photsynthetic dinoflagellate sources for rbcL genes include those from Gonyaulax polyedra (Morse et al. (1995) Science 263: 1522), Amphidinium carterae (Whitney et al. (1998) Aust. J. Plant Physiol. 25: 131), and Symbiodinium (Rowan et al. (1996) Plant Cell 8: 539). A preferred host cell is a strain of photosynthetic bacterium that is transformable and which can be complemented to photoheterotrophic growth by expression of a functional rbcL gene.

III. Shuffling a Form II L subunit from a first species of photosynthetic bacteria with a Form I rbcL subunit from a green algae, cyanobacteria, or a higher plant. The resultant shufflants may be transformed into bacterial host cells which preferably lack endogenous Rubisco activity (e.g., E. coli), algal cells, or plant cells for expression and selection. Phenotype selection of shufflants is typically performed by biochemical assay for RuBP carboxylase and/or RuBP oxygenase activity, such as according to Jordan D B and Ogren W L (1981) op.cit or other suitable assay method selected by the artisan. Example photosynthetic bacteria for the rbcL gene(s) include Rhodobacter sphaeroides (Falcone et al. (1998) J. Bact. 170: 5), Rhodospirrilum rubrum (Falcone and Tabita (1993) J. Bact. 175: 5066; Falcone et al. (1991) J. Bact. 173: 2099) and the like. Example cyanobacteria that can serve as a source of rbcL genes include Synechococcus, Cocochloris peniocystis, and Aphanizomenon flosaquae. Example green algae that can serve as sources of rbcL genes include Euglena gracilis, Chlamadomonas reinhardii, and Anacystis nidulans.

IV. Shuffling a Form I rbcL subunit from a marine algae or green algae with a Form I rbcL subunit from a higher plant species. The resultant shufflants may be transformed into host cells which preferably lack endogenous Rubisco activity but which fold and process higher plant Rubisco subunits correctly for expression and selection, and generally encode and express a complementing rbcS subunit, often from the higher plant species. Suitable host cells can be Synechococcus R2 (Chauvat et al. (1983) Mol. Gen. Genet. 91: 39; Lightfoot et al. (1988) J. Gen. Microb. 134: 1509), Synechocystis (Williams J G K (1988) Meth. Enzymol. 167: 85), or Rubisco-deficient tobacco mutants (e.g., H7 and Sp25; Foyer et al. (1995) J. Exp.

Botany 266: 1445) with the Sp25 mutant of tobacco being useful for rbcL subunit screening. Phenotype selection of shufflants is typically performed by growth selection in a CO₂ incubation environment or on a bicarbonate-containing growth medium, or by biochemical assay for RuBP carboxylase and/or RuBP oxygenase activity, such as according to Jordan D B and Ogren W L (1981) op.cit or other suitable assay method selected by the artisan. Example marine algae for the marine algal rbcL gene(s) include Porphyridium, Olisthodiscus, Cryptomonas, C. fusiformis, or Cylindrotheca N1.

Example higher plants that can serve as a source of rbcL genes include, but are not limited to: Zea mays (C4), Amaranthus hybridus (C4), Glycine max (C3), and Nicotiana tabacum (C3).

V. Shuffling a Form I rbcL subunit from a higher plant with mutagenized variants thereof. An rbcL gene (“parental gene”) from a species of C3 or C4 plant is subjected to mutagenesis and shuffling/selection to generate a population of mutagenized shufflants which have substantial sequence identity to the parental gene. The population of mutagenized shufflants is transferred into a population of host cells wherein the mutagenized shufflants are expressed and the resultant transformed host cell population is selected or screened for an enhanced Rubisco phenotype. Suitable host cells can be Synechococcus (S⁺L⁻; for selecting L gene shufflants, S⁻L⁺; for selecting S gene shufflants) or Rubisco-deficient tobacco mutants (e.g., H7 and Sp25; Foyer et al. (1995) J. Exp. Botany 266: 1445) with the Sp25 mutant of tobacco being useful for rbcL subunit screening. Phenotype selection of shufflants is typically performed by growth selection in a CO₂ incubation environment or on a bicarbonate-containing growth medium, or by biochemical assay for RuBP carboxylase and/or RuBP oxygenase activity, such as according to Jordan D B and Ogren W L (1981) op.cit or other suitable assay method selected by the artisan.

A preferred selection protocol comprises culturing the shufflant transformants as replicate cultures (e.g., replica plates on minimal agar medium) in a plurality of incubation environments wherein the ratio of CO₂/O₂ (or, as a proxy, temperature) is gradually increased and selecting those transformants which exhibit large colony size even at low CO2/O2 ratios. Selected transformants are used to obtain the L gene shufflant sequences and subject them to one or more subsequent rounds of shuffling and selection, optionally including mutagenesis.

### Transcriptional Regulatory Sequences

Suitable transcriptional regulatory sequences include: cauliflower mosaic virus 19S and 35S promoters, NOS promoter, OCS promoter, rbcS promoter, Brassica heat shock promoter, synthetic promoters, non-plant promoters modified, if necessary, for function in plant cells, substantially any promoter that naturally occurs in a plant genome, promoters of plant viruses or Ti plasmids, tissue-preferential promoters or cis-acting elements, light-responsive promoters or cis-acting elements (e.g., rbcS LRE), hormone-responsive cis-acting elements, developmental stage-specific promoters and cis-acting elements, viral promoters (e.g., from Tobacco Mosaic virus, Brome Mosaic Virus, Cauliflower Mosaic virus, and the like), and the like. In a variation, a transcriptional regulatory sequence from a first plant species is optimized for functionality in a second plant species by application of recursive sequence shuffling.

Transcriptional regulatory sequences for expression of shuffled rbcL sequences in chloroplasts is known in the art (Daniell et al. (1998) op.cit; O'Neill et al. (1993) The Plant Journal 3: 729; Maliga P (1993) op.cit), as are homologous recombination vectors.

### Host Cells for Screening rbc Gene Shufflants

A variety of suitable host cells will be apparent to those skilled in the art. Of particular note, Form II rbcL gene shufflants can be expressed in the Cbb Rubisco deletion mutant strain of R. Rubrum and in other bacterial hosts, including E. coli, as well as higher taxonomic host cells. However, Form I subunits from higher plants are not processed correctly in bacterial host cells, so Form I rbcL and rbcS shufflants are generally expressed for Rubisco phenotype screening in Synechococcus mutants, Rubisco-deficient tobacco cells, or the like.

Transformation

The transformation of plants and protoplasts in accordance with the invention may be carried out in essentially any of the various ways known to those skilled in the art of plant molecular biology. See, in general, Methods in Enzymology Vol. 153 (“Recombinant DNA Part D”) 1987, Wu and Grossman Eds., Academic Press, incorporated herein by reference. Additional useful general references for plant cell cloning, culture and regeneration include Jones (ed) (1995) Plant Gene Transfer and Expression Protocols—Methods in Molecular Biology, Volume 49 Humana Press Towata N.J.; Payne et al. (1992) Plant Cell and Tissue Culture in Liquid Systems John Wiley & Sons, Inc. New York, N.Y. (Payne); and Gamborg and Phillips (eds) (1995) Plant Cell, Tissue and Organ Culture; Fundamental Methods Springer Lab Manual, Springer-Verlag (Berlin Heidelberg New York) (Gamborg). A variety of cell culture media are described in Atlas and Parks (eds) The Handbook of Microbiological Media (1993) CRC Press, Boca Raton, Fla. (Atlas). Additional information for plant cell culture is found in available commercial literature such as the Life Science Research Cell Culture Catalogue (1998) from Sigma-Aldrich, Inc (St Louis, Mo.) (Sigma-LSRCCC) and, e.g., the Plant Culture Catalogue and supplement (1997) also from Sigma-Aldrich, Inc (St Louis, Mo.) (Sigma-PCCS). Additional details regarding plant cell culture are found in Croy, (ed.) (1993) Plant Molecular Biology Bios Scientific Publishers, Oxford, U.K. General texts discussing cloning and other techniques relevant to the present invention, in a variety of contexts, include: Berger and Kimmel, Guide to Molecular Cloning Techniques, Methods in Enzymology volume 152 Academic Press, Inc., San Diego, CA (Berger); Sambrook et al., Molecular Cloning—A Laboratory Manual (2nd Ed.). Vol. 1-3, Cold Spring Harbor Laboratory, Cold Spring Harbor, N.Y., 1989 (“Sambrook”) and Current Protocols in Molecular Biology, F. M. Ausubel et al., eds., Current Protocols, a joint venture between Greene Publishing Associates, Inc. and John Wiley & Sons, Inc., (supplemented through 1999) (“Ausubel”)).

As used herein, the term “transformation” means alteration of the genotype of a host plant by the introduction of a nucleic acid sequence. The nucleic acid sequence need not necessarily originate from a different source, but it will, at some point, have been external to the cell into which it is to be introduced.

In one embodiment, the foreign nucleic acid is mechanically transferred by microinjection directly into plant cells by use of micropipettes. Alternatively, the foreign nucleic acid may be transferred into the plant cell by using polyethylene glycol. This forms a precipitation complex with the genetic material that is taken up by the cell (e.g., by incubation of protoplasts with “naked DNA” in the presence of polyethylenelycol)(Paszkowski et al., (1984) EMBO J. 3:2717-22; Baker et al (1985) Plant Genetics, 201-211; Li et al. (1990) Plant Molecular Biology Report 8(4)276-291].

In another embodiment of this invention, the introduced gene may be introduced into the plant cells by electroporation (Fromm et al., (1985) “Expression of Genes Transferred into Monocot and Dicot Plant Cells by Electroporation,” Proc. Natl Acad. Sci. USA 82:5824, which is incorporated herein by reference). In this technique, plant protoplasts are electroporated in the presence of plasmids or nucleic acids containing the relevant genetic construct. Electrical impulses of high field strength reversibly permeabilize biomembranes allowing the introduction of the plasmids. Electroporated plant protoplasts reform the cell wall, divide, and form a plant callus. Selection of the transformed plant cells with the transformed gene can be accomplished using phenotypic markers.

Cauliflower mosaic virus (CaMV) may also be used as a vector for introducing the foreign nucleic acid into plant cells (Hohn et al., (1982) “Molecular Biology of Plant Tumors,” Academic Press, New York, pp. 549-560; Howell, U.S. Pat. No. 4,407,956). CaMV viral DNA genome is inserted into a parent bacterial plasmid creating a recombinant DNA molecule which can be propagated in bacteria. After cloning, the recombinant plasmid again may be cloned and further modified by introduction of the desired DNA sequence into the unique restriction site of the linker. The modified viral portion of the recombinant plasmid is then excised from the parent bacterial plasmid, and used to inoculate the plant cells or plants.

Another method of introduction of nucleic acid segments is high velocity ballistic penetration by small particles with the nucleic acid either within the matrix of small beads or particles, or on the surface (Klein et al., (1987) Nature 327:70-73). Although typically only a single introduction of a new nucleic acid segment is required, this method particularly provides for multiple introductions.

A method of introducing the nucleic acid segments into plant cells is to infect a plant cell, an explant, a meristem or a seed with Agrobacterium tumefaciens transformed with the segment. Under appropriate conditions known in the art, the transformed plant cells are grown to form shoots, roots, and develop further into plants. The nucleic acid segments can be introduced into appropriate plant cells, for example, by means of the Ti plasmid of Agrobacterium tumefaciens. The Ti plasmid is transmitted to plant cells upon infection by Agrobacterium tumefaciens, and is stably integrated into the plant genome (Horsch et al., (1984) “Inheritance of Functional Foreign Genes in Plants,” Science, 233:496-498; Fraley et al., (1983) Proc. Natl. Acad. Sci. USA 80:4803).

Ti plasmids contain two regions essential for the production of transformed cells. One of these, named transfer DNA (T DNA), induces tumor formation. The other, termed virulent region, is essential for the introduction of the T DNA into plants. The transfer DNA region, which transfers to the plant genome, can be increased in size by the insertion of the foreign nucleic acid sequence without its transferring ability being affected. By removing the tumor-causing genes so that they no longer interfere, the modified Ti plasmid can then be used as a vector for the transfer of the gene constricts of the invention into an appropriate plant cell, such being a “disabled Ti vector.”

All plant cells which can be transformed by Agrobacterium and whole plants regenerated from the transformed cells can also be transformed according to the invention so as to produce transformed whole plants which contain the transferred foreign nucleic acid sequence.

There are presently at least three different ways to transform plant cells with Agrobacterium: (1) co-cultivation of Agrobacterium with cultured isolated protoplasts; (2) transformation of cells or tissues with Agrobacterium, or (3) transformation of seeds, apices or meristems with Agrobacterium.

Method (1) uses an established culture system that allows culturing protoplasts and plant regeneration from cultured protoplasts.

Method (2) implies (a) that the plant cells or tissues can be transformed by Agrobacterium and (b) that the transformed cells or tissues can be induced to regenerate into whole plants.

Method (3) uses micropropagation. In the binary system, to have infection, two plasmids are needed: a T-DNA containing plasmid and a vir plasmid. Any one of a number of T-DNA containing plasmids can be used, the main issue being that one be able to select independently for each of the two plasmids.

After transformation of the plant cell or plant, those plant cells or plants transformed by the Ti plasmid so that the desired DNA segment is integrated can be selected by an appropriate phenotypic marker. These phenotypic markers include, but are not limited to, antibiotic resistance, herbicide resistance or visual observation. Other phenotypic markers are known in the art and may be used in this invention.

Protoplast Transformation

Numerous protocols for establishment of transformable protoplasts from a variety of plant types and subsequent transformation of the cultured protoplasts are available in the art and are incorporated herein by general reference. For examples, see Hashimoto et al. (1990) Plant Physiol. 93: 857; Plant Protoplasts, Fowke L C and Constabel F, eds., CRC Press (1994); Saunders et al. (1993) Applications of Plant In Vitro Technology Symposium, UPM, 16-18 Nov. 1993; and Lyznik et al. (1991) BioTechniques 10: 295, each of which is incorporated herein by reference).

All plants from which protoplasts can be isolated and cultured to give whole regenerated plants can be transformed by the present invention so that whole plants are recovered which contain the transferred foreign gene. Some suitable plants include, for example, species from the genera Fragaria, Lotus, Medicago, Onobrychis, Trifolium, Trigonella, Vigna, Citrus, Lilnum, Geranium, Manihot, Daucus, Arabidopsis, Brassica, Raphanas, Sinapis, Atropa, Capsicum, Hyoscyamus, Lycopersicon, Nicotiana, Solanum, Petunia, Digitalis, Majorana, Ciohorium, Helianthus, Lactuca, Bromus, Asparagus, Antirrhinum, Hererocallis, Nemesia, Pelargonium, Panicum, Pennisetum, Ranunculus, Senecio, Salpiglossis, Cucumis, Browaalia, Glycine, Lolium, Zea, Triticum, Sorghum, and Datura.

It is known that practically all plants can be regenerated from cultured cells or tissues, including but not limited to all major cereal crop species, sugarcane, sugar beet, cotton, fruit and other trees, legumes and vegetables. Limited knowledge presently exists on whether all of these plants can be transformed by Agrobacterium. Species which are a natural plant host for Agrobacterium may be transformable in vitro. Although monocotyledonous plants, and in particular, cereals and grasses, are not natural hosts to Agrobacterium, work to transform them using Agrobacterium has also been successfully carried out by numerous investigators (Hooykas-Van Slogteren et al., (1984) Nature 311:763-764; Hernalsteens et al., (1984) EMBO J. 3:3039-41; Byteiber, et al. (1987) Proc. Natl. Acad. Sci. USA: 5345-5349; Graves and Goldman, (1986) Plant Mol. Biol 7: 43-50; Grimsley et al. (1988) Biochemistry 6: 185-189; WO 86/03776; Shimamoto et al. Nature (1989) 338: 274-276). Monocots may also be transformed by techniques or with vectors other than Agrobacterium. For example, monocots have been transformed by electroporation (Fromm et al. [1986] Nature 319:791-793; Rhodes et al. Science [1988] 240: 204-207), direct gene transfer (Baker et al. [1985] Plant Genetics 201-211), by using pollen-mediated vectors (EP 0 270 356), and by injection of DNA into floral tillers (de la Pena et al. [1987], Nature 325:274-276). Additional plant genera that may be transformed by Agrobacterium include Chrysanthemum, Dianthus, Gerbera, Euphorbia, Pelaronium, Ipomoea, Passiflora, Cyclamen, Malus, Prunus, Rosa, Rubus, Populus, Santalum, Allium, Lilium, Narcissus, Ananas, Arachis, Phaseolus and Pisum.

Chloroplast Transformation

As the rbcL gene of higher plants is encoded on the chloroplast genome and expressed in chloroplasts, it is generally useful to transform the shufflant Form I rbcL encoding sequences into chloroplasts if the host cells are derived from higher plants. Numerous methods are available in the art to accomplish the chloroplast transformation and expression (Daniell et al. (1998) op.cit; O'Neill et al. (1993) The Plant Journal 3: 729; Maliga P (1993) op.cit). The rbcL expression construct comprises a transcriptional regulatory sequence functional in plants operably linked to a polynucleotide encoding an enhanced Rubisco protein subunit. With respect to polynucleotide sequences encoding Form I Rubisco L subunit proteins, it is generally desirable to express such encoding sequences in plastids, such as chloroplasts, for appropriate transcription, translation, and processing. With reference to expression cassettes which are designed to function in chloroplasts, such as an expression cassette encoding a large subunit of Rubisco (rbcL) in a higher plant, the expression cassette comprises the sequences necessary to ensure expression in chloroplasts—typically the Rubisco L subunit encoding sequence is flanked by two regions of homology to the plastid genome so as to effect a homologous recombination with the chloroplastid genome; often a selectable marker gene is also present within the flanking plastid DNA sequences to facilitate selection of genetically stable transformed chloroplasts in the resultant transplastonic plant cells (see Maliga P (1993) TIBTECH 11: 101; Daniell et al. (1998) Nature Biotechnology 16: 346, and references cited therein).

### Recovery of Selected Polynucleotide Sequences

A variety of selection and screening methods will be apparent to those skilled in the art, and will depend upon the particular phenotypic properties that are desired. The selected shuffled genetic sequences can be recovered for further shuffling or for direct use by any applicable method, including but not limited to: recovery of DNA, RNA, or cDNA from cells (or PCR-amplified copies thereof) from cells or medium, recovery of sequences from host chromosomal DNA or PCR-amplified copies thereof, recovery of episome (e.g., expression vector) such as a plasmid, cosmid, viral vector, artificial chromosome, and the like, or other suitable recovery method known in the art.

Any suitable art-known method, including RT-PCR or PCR, can be used to obtain the selected shufflant sequence(s) for subsequent manipulation and shuffling.

### Backcrossing

After a desired Rubisco phenotype is acquired to a satisfactory extent by a selected shuffled gene or portion thereof, it is often desirable to remove mutations which are not essential or substantially important to retention of the desired phenotype (“superfluous mutations”). This is particularly desirable when the shuffled gene sequence is to be reintroduced back into a higher plant, as it is often preferred to harmonize the shufflant Rubisco subunit sequence with the endogenous Rubisco subunit sequence in the higher plant taxonomic species genome while retaining the desired Rubisco pheotype obtained from the iterative shuffling/selection process. Superfluous mutations can be removed by backcrossing, which is shuffling the selected shuffled rbcL gene(s) with one or more parental rbcL gene and/or naturally-occurring rbcL gene(s) (or portions thereof) and selecting the resultant collection of shufflants for those species that retain the desired phenotype. The same process may be employed for the rbcS genes. By employing this method, typically in two or more recursive cycles of shuffling against parental or naturally-occurring viral genome(s) (or portions thereof) and selection for retention of the desired Rubisco phenotype, it is possible to generate and isolate selected shufflants which incorporate substantially only those mutations necessary to confer the desired phenotype, whilst having the remainder of the genome (or portion thereof) consist of sequence which is substantially identical to the parental (or wild-type) sequence(s). As one example of backcrossing, a pea Rubisco subunit gene (small subunit) can be shuffled and selected for the capacity to substantially function in any Angiosperm plant cells; the resultant selected shufflants can be backcrossed with one or more Rubisco genes of a particular plant species and selected for the capacity to retain the capacity to confer the phenotype. After several cycles of such backcrossing, the backcrossing will yield gene(s) which contain the mutations necessary for the desired phenotype, and will otherwise have a genomic sequence substantially identical to the genome(s) of the host genome.

Isolated components (e.g., genes, regulatory sequences, replication origins, and the like) can be optimized and then backcrossed with parental sequences so as to obtain optimized components which are substantially free of superfluous mutations.

### Transgenic Hosts

Transgenes and expression vectors to express shufflant rbc sequences can be constructed by any suitable method known in the art; by either PCR or RT-PCR amplification from a suitable cell type or by ligating or amplifying a set of overlapping synthetic oligonucleotides; publicly available sequence databases and the literature can be used to select the polynucleotide sequence(s) to encode the specific protein desired, including any mutations, consensus sequence, or mutation kernal desired by the practitioner. The coding sequence(s) are operably linked to a transcriptional regulatory sequence and, if desired, an origin of replication. Antisense or sense-suppression transgenes and genetic sequences can be optimized or adapted for particular host cells and organisms by the described methods.

The transgene(s) and/or expression vectors are transferred into host cells, protoplasts, pluripotent embryonic plant cells, microbes, or fungi by a suitable method, such as for example lipofection, electroporation, microinjection, biolistics, Agrobacterium tumefaciens transduction of Ti plasmid, calcium phosphate precipitation, PEG-mediated DNA uptake, electroporation, electrofusion, or other method. Stable transfectant host cells can be prepared by art-known methods, as can transgenic cell lines.

Target Plants

As used herein, “plant” refers to either a whole plant, a plant part, a plant cell, or a group of plant cells. The class of plants which can be used in the method of the invention is generally as broad as the class of higher plants amenable to protoplast transformation techniques, including both monocotyledonous and dicotyledonous plants. It includes plants of a variety of ploidy levels, including polyploid, diploid and haploid, and may employ non-regenerable cells for certain aspects which do not require development of an adult plant for selection or in vivo shuffling.

As noted, preferred plants for the transformation and expression of Rubisco include agronomically and horticulturally important species. Such species include, but are not restricted to members of the families: Graminae (including corn, rye, triticale, barley, millet, rice, wheat, oats, etc.); Leguminosae (including pea, beans, lentil, peanut, yam bean, cowpeas, velvet beans, soybean, clover, alfalfa, lupine, vetch, lotus, sweet clover, wisteria, and sweetpea); Compositae (the largest family of vascular plants, including at least 1,000 genera, including important commercial crops such as sunflower) and Rosaciae (including raspberry, apricot, almond, peach, rose, etc.), as well as nut plants (including, walnut, pecan, hazelnut, etc.).

Targets for the invention also include plants from the genera: Agrostis, Allium, Antirrhinum, Apium, Arachis, Asparagus, Atropa, Avena (e.g., oats), Bambusa, Brassica, Bromus, Browaalia, Camellia, Cannabis, Capsicum, Cicer, Chenopodium, Chichorium, Citrus, Coffea, Coix, Cucumis, Curcubita, Cynodon, Dactylis, Datura, Daucus, Digitalis, Dioscorea, Elaeis, Eleusine, Festuco, Fragaria, Geranium, Glycine, Helianthus, Heterocallis, Hevea, Hordeum (e.g., barley), Hyoscyamus, Ipomoea, Lactuca, Lens, Lilium, Linum, Lolium, Lotus, Lycopersicon, Majorana, Malus, Mangifera, Manihot, Medicago, Nemesia, Nicotiana, Onobrychis, Oryza (e.g., rice), Panicum, Pelargonium, Pennisetum (e.g., millet), Petunia, Pisum, Phaseolus, Phleum, Poa, Prunus, Ranunculus, Raphanus, Ribes, Ricinus, Rubus, Saccharum, Salpiglossis, Secale (e.g., rye), Senecio, Setaria, Sinapis, Solanum, Sorghum, Stenotaphrum, Theobroma, Trifolium, Trigonella, Triticum (e.g., wheat), Vicia, Vigna, Vitis, Zea (e.g., corn), and the Olyreae, the Pharoideae and many others.

Common crop plants which are targets of the present invention include corn, rice, triticale, rye, cotton, soybean, sorghum, wheat, oats, barley, millet, sunflower, canola, peas beans, lentils, peanuts, yam beans, cowpeas, velvet beans, clover, alfalfa, lupine, vetch, lotus, sweet clover, wisteria, sweetpea and nut plants (e.g., walnut, pecan, etc).

Regeneration

Normally, regeneration will be involved in obtaining a whole plant from the transformation process. The term “transgenote” refers to the immediate product of the transformation process and to resultant whole transgenic plants.

The term “regeneration” as used herein, means growing a whole plant from a plant cell, a group of plant cells, a plant part or a plant piece (e.g. from a protoplast, callus, or tissue part).

Plant regeneration from cultural protoplasts is described in Evans et al., “Protoplasts Isolation and Culture,” Handbook of Plant Cell Cultures 1: 124-176 (MacMillan Publishing Co. New York 1983); M. R. Davey, “Recent Developments in the Culture and Regeneration of Plant Protoplasts,” Protoplasts, (1983)—Lecture Proceedings, pp. 12-29, (Birkhauser, Basal 1983); P. J. Dale, “Protoplast Culture and Plant Regeneration of Cereals and Other Recalcitrant Crops,” Protoplasts (1983)—Lecture Proceedings, pp. 31-41, (Birkhauser, Basel 1983); and H. Binding, “Regeneration of Plants,” Plant Protoplasts, pp. 21-73, (CRC Press, Boca Raton 1985).

Additional details regarding plant regeneration are found in Jones (ed) (1995) Plant Gene Transfer and Expression Protocols—Methods in Molecular Biology, Volume 49 Humana Press Towata N.J.; Payne et al. (1992) Plant Cell and Tissue Culture in Liquid Systems John Wiley & Sons, Inc. New York, N.Y. (Payne); Gamborg and Phillips (eds) (1995) Plant Cell, Tissue and Organ Culture, Fundamental Methods Springer Lab Manual, Springer-Verlag (Berlin Heidelberg New York) (Gamborg) and in Croy, (ed.) (1993) Plant Molecular Biology.

Regeneration from protoplasts varies from species to species of plants, but generally a suspension of transformed protoplasts containing copies of the exogenous sequence is first made. In certain species embryo formation can then be induced from the protoplast suspension, to the stage of ripening and germination as natural embryos. The culture media will generally contain various amino acids and hormones, such as auxin and cytokinins. It is sometimes advantageous to add glutamic acid and proline to the medium, especially for such species as corn and alfalfa. Shoots and roots normally develop simultaneously. Efficient regeneration will depend on the medium, on the genotype, and on the history of the culture. If these three variables are controlled, then regeneration is fully reproducible and repeatable.

Regeneration also occurs from plant callus, explants, organs or parts. Transformation can be performed in the context of organ or plant part regeneration. See, Methods in Enzymology, supra; also Methods in Enzymology, Vol. 1118; and Klee et al., (1987) Annual Review of Plant Physiology, 38:467-486.

In vegetatively propagated crops, the mature transgenic plants are propagated by the taking of cuttings or by tissue culture techniques to produce multiple identical plants for trialling, such as testing for production characteristics. Selection of desirable transgenotes is made and new varieties are obtained thereby, and propagated vegetatively for commercial sale.

In seed propagated crops, the mature transgenic plants are self crossed to produce a homozygous inbred plant. The inbred plant produces seed containing the gene for the newly introduced foreign gene activity level. These seeds can be grown to produce plants that would produce the selected phenotype.

The inbreds according to this invention can be used to develop new hybrids. In this method a selected inbred line is crossed with another inbred line to produce the hybrid. The offspring resulting from the first experimental crossing of two parents is known in the art as the F1 hybrid, or first filial generation. Of the two parents crossed to produce F1 progeny according to the present invention, one or both parents can be transgenic plants.

Parts obtained from the regenerated plant, such as flowers, seeds, leaves, branches, fruit, and the like are covered by the invention, provided that these parts comprise cells which have been so transformed. Progeny and variants, and mutants of the regenerated plants are also included within the scope of this invention, provided that these parts comprise the introduced DNA sequences. Progeny and variants, and mutants of the regenerated plants are also included within the scope of this invention.

Shuffling Rubisco, the Calvin Cycle Operon and Other Genes for Cyanobacterial CO₂ Production and for Production of Useful Chemicals and Fuels

The development of technologies for effective biological fixation of CO₂ on a global scale can mitigate the effects of atmospheric greenhouse gas emission. Cyanobacterial aquaculture (‘cyanofarming’) offers one of the most productive solutions for global greenhouse gas control, as compared to other biological alternatives aimed at CO₂ fixation (plants, microscopic eukaryotic algae, or non-photosynthetic organisms).

Cyanofarming has shown that photosynthetic bacteria are the most promising and productive biosystem in terms of stoichiometric CO₂ fixation into biomass, per photon utilized, per mole of water required, per unit of area of land required. However, to become a viable CO₂ abatement technology for global use, current biomass productivity of cyanofarming has to be improved by an estimated 10-20 fold.

This can be accomplished in the context of the present invention by engineering and evolving highly productive and robust cyanobacterial strains for shallow pond bioprocessing, specifically by engineering rubisco, calvin and krebs cycle enzymes and other genes as discussed below. Shuffling of genomic targets, such as Rubisco, impacts the overall efficiency of CO₂ fixation and biomass productivity of cyanobacteria.

DNA-shuffling based evolutionary technologies are used to shuffle rubisco (ribulose 1,5-bisphosphate carboxylase/oxygenase). In addition, the Calvin or Krebs cycle operons can be shuffled in its entirety to further enhance CO₂ fixation/biomass production. For example, the inclusion of the Calvin cycle (cbb) operon as a genomic target for heterologous expression in cyanobacteria and for shuffling to optimize performance can be conducted in concert with Rubisco shffling or independent from Rubisco shuffling. A “Calvin cycle enzyme” herein is an enzyme which is normally active in the Calvin cycle (e.g., Rubisco). A “Krebs cycle enzyme” herein is an enzyme which is normally active in the Krebs cycle. In the present invention, Calvin and Krebs cycle enzymes, and their homologues, are shuffled to produce new enzymes and enzyme pathways with elevated levels of carbon fixation.

Both growth yield and rate of cyanobacteria on CO₂ fixation is dependent on the nature and effiency of the biosynthesis of reduced carbon compounds by the cells. In biosynthetic pathways for generation of useful carbon storage compounds, targets include genes involved in control of intracellular acetate pool and synthesis of a nitrogen-free intracellular storage compounds, such as poly(hydroxybutyrate) (PHB). Other genomic targets (e.g. carbonate transport proteins, stress, salinity or chemical tolerance genes) can also be examined and modified on as needed basis. Evolution of the targets by recursive molecular breeding in-vitro provides architectural foundation for subsequent construction of the desired highly productive cyanobacterial strains for large-scale CO₂, fixation in various distinct cyanofarming settings (climate, water chemistry/salinity).

To create an economic incentive to practice sustainable CO₂ fixation-based bioprocesses (that ultimately may become less vulnerable to greenhouse gas abatement, economics and regulations), cyanofarming as a technology utilizes processes aimed at manufacturing of value-added products, including renewable fuels, whether originating directly from metabolism of cyanobacterial cells, or obtained in a secondary cyanobiomass processing.

The primary group of technical objectives (assimilatory CO₂ metabolism) targets development of prototype cyanobacterial strains with high productivity and fast autotrophic growth under non-limiting CO₂ conditions. The strains which are produced can be used for large-scale commercial cyanofarming with a significant contribution to atmospheric CO₂ abatement (providing CO₂ credit generation).

The secondary group of technical objectives is dedicated to achieving enhanced production in the prototype cyanobacterial strains of non-carbohydrate intracellular carbon storage compounds so that the Joule (BTU) content of the biomass is increased and the nitrogen content is decreased. This area is recognized as very likely to be a technology component (a) for increasing overall CO₂-fixing productivity of cyanofarming, (b) for increasing recoverable added value from output of cyanobacterial autotrophic growth, and (c) for control of NOₓ emissions from combustion of cyanobacterial biomass. Time and scale of deployment of efforts in the secondary group of technical objectives is contingent on experimental results obtained in the primary group of objectives.

Cyanobacteria as Targets for Organism Engineering and Evolution

The understanding of genomics in cyanobacterial biology is very good. Extensive taxonomic studies have been published, and many characterized species exist in accessible collections. Whole genome sequencing has been completed for Synechocystis, and several other strains and species are being sequenced. Molecular biology tools are well developed for cyanobacteria. Recombinant DNA transformation efficiency is very good, a range of mutants for laboratory manipulations required for strain development are available, and characterized cyanobacterial expression vectors exist. A significant body of knowledge exists in cyanobacterial enzymology and genomics pertinent to central metabolism, photosynthesis, CO₂ transport, nitrogen fixation, stress-factor resistance and secondary metabolite production (e.g. polyhydroxyalkanoates, carotenoids, extracellular toxins).

Significantly, cyanobacterial rubisco can be functionally expressed in other bacterial hosts (including E. coli). Rubisco is a target for DNA shuffling based evolutionary developments aimed to tailor/optimize kinetic parameters of this enzyme (t, Vₘₐₓ) which are factors that affect overall metabolic productivity of the cyanobacterial cells and thus are of utmost importance for CO₂-fixation based biomass production. HTP assay technology for Rubisco evolution is straightforward (based on use of ¹⁴C carbonate as set forth supra). Development of growth-based selection systems for sampling large shuffled libraries is highly feasible.

Cyanobacterial Growth Productivity Compared to CO₂ Emissions of Coal-Firing Power Plant

A nominal 0.45 GW coal-firing power plant produces ˜100,000 T of CO₂, per year, or ˜275 T of CO₂ per day, which is equivalent to 75 T of carbon per day. To capture all of this 75 T/day amount of CO₂ in a photosynthetic bioprocess, ˜150 T of dry biomass are produced daily (based on ˜50% carbon content typical for cyanobacterial and bacterial biomass). Based on the disclosed data for average year around productivities at commercial cyanobacterial farms for Spirulina (Arthrospira) species in Hawaii, California and India, 4 to 12 grams per m² per day of dry cell biomass can be reliably produced (whether using basified and carbonated sea water or artificial brackish alkaline carbonated water as medium). This productivity figure is based on calculations for shallow (10-20 cm deep) artificial ponds with producing surfaces in the 80-100 acre (32-40 ha) range. At the lower end of the productivity figure, 1 ha of pond area can fix 20 kg/day of carbon and produce 40 kg/day of dry biomass. This means that approximately ˜3750 ha (˜37.5 km²) of pond area are used to fix all of the 75 T of carbon. Thus, an unrealistically high pond area is needed for unmodified strains to fix sufficient carbon to accomidate industrial CO₂ production.

Theoretical yields for Spirulina productivity have been discussed in the literature at 40 grams per m² per day of dry cell biomass (of a standing crop, before light limitation becomes limiting), i.e., roughly 10× that of unmodified strains. This productivity have not been achieved in practice. As cyanobacterial production is improved by optimizing growth conditions, and by shuffling and breeding the cyanobacterial strains to achieve yields close to the theoretical light-dependent limit (˜10 fold improvement in biomass-producing productivity), then ˜375 ha (˜3.75 km²) of ponds will capture the CO₂ output by an ‘average’ coal-firing power plant.

Improvement of productivity beyond the above theoretical figure is attained if cyanobacterial strains are evolved to grow significantly faster (e.g. doubling time in the range of 2-3 hours), under essentially continuous conditions providing for continuous removal of accumulated biomass prior to prevent light limitation requirements in high density cultures. Maintaining such growth rate during night time is not acheived without artificial illumination due to oxygen depletion/anoxic conditions leading to die-off of the cyanoculture.

A partial CO₂ capture processes results in a significant reduction in land needs, controlling facility area to a manageable plot. For example, a 1 km² of cyanofarm, with improved biomass productivities at ˜10× of current, would allow to capture ˜20 T of carbon per day, which is equivalent to ˜25% of the total CO₂ output of an average 0.45 GW power plant.

A goal of the shuffling approaches herein is to develop Cyanobacterial processes for generating reduced carbon compounds in prokaryotic biomass with lowered nitrogen content, which can be used as fuel.

Concurrent with shuffling Rubisco and Calvin cycle enzymes, other uses of cyanobacterial biomass can be shuffled and selected for to simultaneously provide many economically attractive products (i.e., products other than renewable high BTU content fuel production), including soil improvement/fertilizer (and restoration of humic content of eroded topsoil), animal feed (using Spirulina and other non-toxic species to produce very high protein content production of as much as ˜70%), cyanobiomass processing for ethanol and other solvents, biogas production, production of non-food and feed chemicals through metabolic engineering and evolutionary optimization of biosynthetic pathways in cyanobacteria (by DNA shuffling-tailored chemical output). For example, for tailored chemical output, squalene and other non-volatile hydrophobic terpenoids (e.g. steranes) can be produced for technical uses (lubricants), and biopolymers such as polyhydroxybutyrate (primarily for monomer recovery through biomass processing), 3-hydroxybutyrate and crotonate can be produced. Production of protein enriched in high value aminoacids (e.g. phenylalanine) and cyanobiomass processing for aminoacid recovery, carotenoids, tocopherols (antioxidants) can also be produced. Details on these shuffling strategies are set forth below.

Cyanobacterial Productivity Considerations Related to Other CO₂-Fixing Bioprocesses

Among various autotrophic and non-autotrophic systems, microscopic eukaryotic algae closely approach cyanobacteria in their space-time CO₂ fixing capability and biomass productivity. While not as desirable a target as cyanobacteria due to the relatively undeveloped state of eukaryotic algal genomics and biochemistry, eukaryotic microscopic algae are an example secondary target system for shuffling as described herein for cyanobacteria.

Typical agricultural crop plants are inferior to cyanobacteria in CO₂ fixation (˜5-10 fold). Trees are the best land plants for fixing carbon (1-4 T per ha per year). Cyanobacteria such as spirulina fix ˜6.3 T/ha per year; it also produces 16.8 T/ha per year of oxygen (about twice as much as trees). However, crop plants, which are grown for a variety of purposes, can also be shuffled for improved CO₂ fixation.

In respect to protein production, spirulina is ˜20 times more efficient than soybean and ˜40 times more efficient than corn. Cyanobacteria do not require fertile land. Growing cyanobacterial protein requires 4-7 times less water than soybean and corn. Presence of pyocyanin pigment in photosynthetic systems of cyanobacteria makes overall biomass yield is 2-5 times higher, than in soybean and corn, on per photon basis. Thus, shuffling to achieve protein biomass production is attractively practiced in cyanobacteria. However, crop plants, which are grown for a variety of purposes, can also be shuffled for improved protein production according to the present invention.

State-of-the-art commercial cyanofarming (aimed primarily on spirulina production for food) provides invaluable information and validated practical experience in such technology components as hardware and process design/engineering, biomass separation and drying, as well as in-depth insights into many other related technical problems (managing weed species, maintenance continuous year around cultivation). Sources describing cyanofarming include: Microalgae of Economic Potential by A. Richmond in CRC Handbook of Microalgal Mass Culture, 1986, CRC Press, Boca Raton, Fla.; Microalgae: Organic Factories of the Future. Cyanotech Corp. 1998. and other information from Cyanotech: http://www.cyanotech.com; Spirulina: Environmental Advantages; Earthrise Farms, California: http://spirulina.com/SPPEnvironment.html; Jeeji Bai N (Poster Abstract, 1995) “Decentralized Arthrospira (“Spirulina”) culture facility for income generation in rural areas” 1992 data. Shrii A.M.M Mudragappa Chettiar Research Centre, Tharamani, Madras 600113, India; Alkalophilic cyanobacteria: digests of Curds et al, 1986 and Finlay et al, 1987 works http://www.nhm.ac.uk/zoology/extreme.html#alk; Spirulina—Production and Potential by Ripley D. Fox 1996. Pub. by Editions Edisud, La Calade, R.N.7 !3090 Aix-en-provice, France; and information and references cited at http://www.cyanosite.bio.purdue.edu.

Experimental Approach

The success of cyanobacterial CO₂ bioprocess development and practical applications include a recognition of the principal bottlenecks which limit overall productivity of biomass with desired properties. According to available literature data, cyanobacterial growth productivity in today's art typically reach only about 10%-15% of theoretical limits (before light limitations in open systems are reached). It is apparent that significant improvements both in (i) primary assimilatory metabolism of CO₂, and in (ii) biosynthesis of reduced carbon compounds, increase volumetric productivity, and accelerate autotrophic growth.

Improvement of the later feature of production strains of cyanobacteria is particularly useful, as it overcomes usual “theoretical” limitations based on calculations of a “standing crap” due to light limitations. There is overall “reducing overcapacity” generated by photosynthetic bioenergetics in cyanobacteria, as compared to that of “assimilatory capacity” of carbon flux. Improvement of the carbon flux during autotrophic growth is achieved by molecular breeding of several target genes in cyanobacterial genome, as well by introduction and molecular breeding of additional sets of heterologous genes which are known to play critical role in biomass production and biomass composition.

The primary group of technical objectives (assimilatory CO₂ metabolism) targets development of prototype cyanobacterial strains with high productivity and fast autotrophic growth under non-limiting CO₂ conditions. The strains that can be used for large-scale commercial cyanofarming with significant contribution to atmospheric CO₂ abatement (CO₂ credit generation).

The secondary group of technical objectives is dedicated to achieving enhanced production in the prototype cyanobacterial strains of non-carbohydrate intracellular carbon storage compounds so that the Joule (BTU) content of the biomass is increased and the nitrogen content is decreased. This area is recognized as a technology component (a) for increasing overall CO₂-fixing productivity of cyanofarming, (b) for increasing recoverable added value from output of cyanobacterial autotrophic growth, and (c) for control of NOₓ emissions from combustion of cyanobacterial biomass. Time and scale of deployment of efforts in the secondary group of technical objectives is contingent on experminental results obtained in the primary group of objectives.

Shuffling and Organism Engineering for Cyanobacterial Process of CO₂ Fixation: Defining Target Genes for Evolution by Molecular Breeding

Different bottlenecks occur throughout CO₂ flux. These bottlenecks are addressed in a systematic fashion, to achieve optimum performance of the entire cell.

The following, individually and together are targets for shuffling to improve CO₂ fixation: Rubisco sequences encoding large and small subunits and promoter sequences as a primary gate for CO₂ assimilation, the primary assimilatory metabolism via evolution of the Calvin cycle in its functional entirety, and carbon depository biosynthesis of secondary metabolites.

Rubisco as a Putative Bottleneck in Primary CO₂ Assimilatory Metabolism in Cyanobacteria and Rubisco Shuffling

Natural rubisco is a relatively slow enzyme. In the present invention, rubisco is a target for shuffling because the enzyme is a bottleneck in the primary CO₂ assimilatory metabolism in cyanobacteria.

Bacterial rubisco systems known in cyanobacteria and many other autotrophic bacteria are representative enzymes of the L₈S₈ type. Related genes from many accessible organisms are known, constituting a diverse family of homologous genes suitable for family DNA shuffling in vitro. Molecular breeding of rubisco in cyanobacteria provides for tailoring and improvement of this enzyme for increasing catalytic turnover under non-limiting CO₂ concentrations (Vₘₐₓ for CO₂). In the operational practice of cyanofarming, non-limiting CO₂ conditions are easily attained by excess supply of CO₂ (“carbonation on demand”) in the form of sodium bicarbonate buffer (at, or above, 5% of CO₂ equivalents).

Molecular breeding of rubisco for operation under high CO₂ conditions achieves, e.g., “simple” Vₘₐₓ increases in respect to CO₂. Improvement in substrate specificity properties (t) for discrimination between CO₂ and O₂ becomes less important as the need for effective scavenging of low and limiting CO₂ amounts (e.g. at the natural CO₂ abundance level of 0.03-0.04%) in the presence of vast excess (3-4 orders of magnitude) of dioxygen is no longer of significance.

Also, in the presence of large excess of CO₂, minor formation of phosphoglycolate as oxygenation product also be no longer of significance. Furthermore, less significant misfire product issues in rubisco catalytic cycle are effectively addressed by default where the selection and screening of shuffled libraries employs an adequate quantitative measure of incorporated CO₂ in biomass. This technique is readily attained by using C”⁴ carbonate with subsequent quantitative determination of radioactivity associated with cell biomass during screening of shuffled rubisco libraries, where biomass and aqueous medium are separated (e.g. centrifugation in 96 well plates with 2-3 cycles of cell wash by non-radioactive medium or aqueous acid). Experiments performed so far for rubisco assays in vivo (in E. coli) indicate that this assay approach is satisfactory.

Introduction and Molecular Breeding of the Bacterial Calvin Cycle Genes from Organoautrotrophic Organisms (cbb Operons).

Detailed studies in molecular genetics and physiology of autotrophic growth of methylotrophic bacteria have been recently published. Work conducted on Alcaligenes euthrophus H16 (minireview by Bowien at al, 1996 in Microbial Growth on C₁ compounds, p 102-109. and Xantobacter flavus (minireview by Meijer, 1996, in Microbial Growth on C₁ Compounds, 118-125) suggest that the activity of enzymes other than those unique (rubisco and PGK) to the Calvin cycle should also be increased in order to achieve optimal rates of carbon dioxide fixation required for autotrophic growth.

Several complete cbb (Calvin cycle) operons have been identified and completely sequenced at present. The A. euthrophus strain has two fully suitable for molecular breeding in family shuffling (˜15 kb clusters with sequence identity 95%), one is a chromosomal set, the other is plasmid-borne. Both cbb operons are controlled by cbbR transcriptional activator protein (typical representative of LysR family), although the chemical nature of cbbR activator has not been established (not CO₂). Both cbb sets also include cbbZ—2-phosphoglycolate phosphatase (which acts on the product formed by rubisco oxygenation). This is a clear genetic manifestation of the metabolic interaction between the Calvin cycle and oxidative glycolate pathway.

The cbb operons employ isoenzymes of fructose-1,6-bisphosphatase, fructose-1,6-bisphosphate aldolase, transketolase, glycero-3-phosphate dehydrogenase, pentose-5-phosphate epimerase, and several pertinent promoters. Some of these enzymes have unique kinetic and stability properties distinct from non-Calvin cycle chromosomally encoded isoenzymes. Cyanobacterial genes encoding the Calvin cycle enzymes are spread throughout genome, not clustered; thus straightforward in-vitro shuffling of these genes for optimal and balanced performance in concert is relatively difficult. Thus, an experimental approach based on molecular breeding application to the above noted heterologous cbb operons is used, in which these operons or shuffled progeny thereof are expressed in cyanobacteria.

Carbon Storage Compounds in Cyanobacterial CO₂ Fixation

The importance of biosynthesis of reduced carbon compounds during photoautothropic growth is substantial. The nature and the operational efficiency of pathways responsible for cellular production of reduced carbon compounds are critical for overall CO₂ fixation process, both from standpoint of growth rate and volumetric productivity, and from standpoint of ultimate economics of cyanobacterial CO₂ abatement effort which may or may not leverage from value added chemical output in produced biomass.

Ultimately, stoichiometry of metabolic pathways involved in bioconversion of CO₂ and the bioenergetics of cyanobacterial photosynthesis are intricately intertwined with the biosynthetic machinery which produces secondary metabolic products, which serve as strategic or tactical cellular depositories of reduced carbon, whether nutritional, structural or non-functional.

Furthermore, genetic manipulations aimed at increasing carbon flux through the biosynthetic pathways to carbon storage compounds achieves a metabolic situation equivalent to “carbon starvation” during autotrophic growth by effective and (quasi)irreversible carbon sequestration away from the central pathways to insoluble species. This helps alleviate such metabolic flux control problems as product inhibition typically encountered in most enzymes of the Calvin cycle and of other central pathways, including the Krebs cycle (the encoding genes of which are also a target for shuffling in the present invention, in conjunction with those of the Calvin cycle and rubisco).

Biomass rich in reduced carbon compounds (but not nitrogen rich) is ultimately desired for CO₂ abatement and renewable fuel generation. The following technical elements also address these issues.

Controlling Acetate Pool in Cyanobacteria

Metabolic levels of cellular acetyl CoA in bacteria are relevant for channeling carbon flux from the Calvin cycle towards desired carbon storage compounds. Cyanobacteria normally do not produce high levels of acetate/acetyl-CoA and their primary carbon storage compounds are polysaccharides (glycogen). The later are less desirable low value compounds from the standpoint of cyanobacterial biomass value and utilization as they are difficult to process into high quality fuel or chemical output. Polysaccharides are also readily biodegradable, limiting possible non-fuel uses of cyanobacterial biomass for carbon dioxide abatement, such as in soil imporvement applications.

Recent publications (Deng, Coleman, 1999 AEM 65(2):523-8) demonstrate that cyanobacterial metabolism can be at least partially re-routed towards acetyl-CoA dependent secondary metabolite production, namely, ethanol production. Expression of pyruvate decarboxylase (pdc) and alcohol dehydrogenase II (adh) from Zymomonas mobilis in Synechococcus sp. PCC 7942 effectively allowed ethanol production under photosynthetic conditions, albeit at relatively low levels. This work shows successful manipulation of cyanobacterial metabolism towards biosynthetic production of acetate-depended chemical output under autotrophic conditions.

Additional Choices of “Carbon Sink” Pathways for Cyanobacterial CO₂ Fixation Process

The feasibility of enhancing the biosynthesis of polyhydroxybutyrates in cyanobacteria has been demonstrated. Narato, et al, 1998 (Proc. Int. Symp. on Biol. PHAs, 1998, P2) reported Tn5-mutant strain of Synechococcus deregulated in PHB production and thus capable of producing the polymer under nitrogen-sufficient conditions with a rate exceeding that of the wild type. Synechococcus expressing the Alcaligenes pha genes have been reported to accumulate up to 30% of PHB polymer (Akiyama et al, 1998, ibid, P4), and the pha genes have been well maintained without antibiotic selection. Synechocystis strains also possesses own (indigenous) sets of functional polyhydroxybutyrate synthase genes encoding a two-component enzyme which is different from other bacterial PHB synthases.

Accumulation of granular PHB in cyanobacterial cells provides an opportunity for simple and efficient collection of biomass: PHB is heavier than water and mature harvest can be collected simply by gravity sedimentation of cells in the absence of active water flow (e.g. collection pond or tank). PHB (C₄H₆O₂)ₙ has significant Joule/BTU value (approaching that of ethanol); thus, it is attractive as a fuel. If developed initially for CO₂ fixation to form biofuels, processing of cyanobacterial PHB stream can be further developed for higher value applications (e.g. for 3-hydroxybutyrate monomer, 3-hydroxybutyrate oligoesters, and particularly, for crotonic acid, suitable for chemical production of biodegradable and non-biodegradable polymers and co-polymers).

Terpenoids as Chemical Output of Cyanobacterial CO₂ Fixation Process

Various cyanobacteria produce many different terpenoids. From an economic standpoint, only a few higher terpenoids represent significant opportunities for production in open systems, due to the inheritant volatility of C₁₀-C₁₅ compounds. A plethora of cyanobacterial carotenoids (tetraterpenoids) are well known, and cyanobacterial genes catalyzing last committed steps of carotenoid biosynthesis are known.

While carotenoids are high value chemical products used as food colorants and antioxidants, in terms of gross carbon amount, carotenoid market represent a minuscule fraction when compared to CO₂ emissions by power-generating industry. On the other hand, all cyanobacterial species produce various amounts (usually very low) of triteprenes, represented typically by glycosylated bacteriohopanoids. The Synechocystis gene for squalene-hopene cyclase is known. This indicates that Synechocystis and other cyanobacterial species possess a fully functional teprenoid biosynthesis pathway which includes hydrocarbon squalene (C₃₀) as one of the intermediates. Squalene represent a very interesting product both as fuel and as a high quality technical lubricant (with properties superior to lanolin and many synthetic compositions). Lubricant properties of hopanoids are similar to lanolin, and in fact, mixtures of hopanoids are typical and abundant in many petroleum derived lubricants as they are one of the most prominent molecular fossils conserved during diagenesis of petroleum deposits.

Cyanobacteria, as well as most of other bacteria, use a mevalonate-independent pathway for terpenoid biosynthesis. This carbohydrate-dependent pathway. The pathway is believed to have a complex regulation mechanism, and the relevant genes are clustered in a particular sector of genome as a distinct operon (spread throughout genome). Shuffling of a terpenoid output pathway, as an alternative to PHB, is optionally performed.

Proposed development in this direction considers two distinct biosynthetic alternatives for hydrocarbon biosynthesis: (a) breeding genes of the new non-mevalonate pathway, which will require detailed functional genomic study for identification of all relevant genes, or (b) metabolic reconstruction of classical mevalonate-dependent pathway in cyanobacteria. All genes of the mevalonate pathway are known from variety of organisms (including a complete set from yeast and partial sets from bacteria and higher eukaryotes). Moreover, the lower mevalonate pathway and PHB biosynthesis pathway share a set of common genes for committing carbon to acetate and acetoacetyl-CoA. Enabling higher value terpenoid outputs from cyanobacterial CO₂ fixation can impact economics of large-scale cyanofarming applications.

The following example is given to illustrate the invention, but are not to be limiting thereof.

### EXAMPLE 1

### Shuffling of Prokaryotic Form II Rubisco

Rubisco genes of prokaryotes are composed of only the large subunit and are called Form II enzymes. These are present in organisms like Rhodobacter, Thiobacillus, dinoflagellates etc. (Watson G M F and Tabita F (1997) FEMS Microbiology Letters 146: 13-22). A number of Form II Rubisco have been cloned and sequenced and are accessed from gene bank (Robinson et. al J. Bacteriol. 180: 1596-99). Primers are designed for these genes based on consensus sequences and genes from various organisms are isolated as described in literature (Robinson et al). Alternately, all of the genes are synthesized.

The Form II genes from various prokaryotes and dinoflagellates (Morse et al. (1995) Science 268: 1622-1624, Rowan et al. (1996) The Plant Cell 8: 539-553) display high degree of homology are shuffled according to the method of the invention. Briefly, this procedure involves random fragmentation of the genes with DNAse I and selecting nucleotide fragments of 100-300 bp. The fragments are reassembled based on sequence similarity by primeness PCR. Recombination as well as variable levels of mutations that are introduced by the PCR reaction generate the diversity. The assembled genes are cloned into a Rhodospirillum rubrum strain in which the Rubisco gene has been deleted (cbbM mutants, Falcone D L and Tabita F R (1993) J. Bacteriol. 175: 5066-5077). Such strain is either obtained from the laboratory of the authors or is created as described in the publication above. Rhodospirillum rubrum transformation protocols are used as described (Fitzmaurice W P and Roberts G P (1991) Arch. Microbiol 156: 142-144 and Falcone D L op.cit). CbbM mutants are unable to grow autotrophically unless complemented with a functional Form II Rubisco from the shuffled gene pool. Those displaying growth are further screened for a better enzyme with respect to carbon fixation based on their rate of growth. Form II enzymes are unstable under oxygen and do not fix carbon. However dinoflagellate enzymes may be able to sustain some activity under low levels of oxygen (Whitney S M and Andrews T J 1998, 25: 131-138). Transformed R. rubrum containing various functional Form II Rubisco genes from shuffled library can be grown in the presence of different levels of oxygen. Those displaying growth can be presumed to contain oxygen-tolerant enzymes. The oxygen stability is gauged based on the ability to grow under different concentrations of oxygen.

Colonies expressing shuffled Form II Rubisco are grown in larger amounts in liquid culture and assayed for carboxylation reaction in the presence of various oxygen concentrations as described (Whitney S M and Andrews T J 1998, 25: 131-138). The extent of carboxylation in the presence of oxygen is quantitated.

Cyanobacterial Rubisco resemble those of higher plant forms in that they are composed of small and large subunits assembled into a hexadecimeric holoenzyme. The two subunits are coded by rbcS and rbcL genes. These genes have been functionally expressed in E. coli (Tabita F R and Small C L 1985. PNAS 82: 6100-6103, van der Vies S M et al. The EMBO Journal 5.2439-2444). Both these genes are isolated and cloned in E. coli by described methods. Various L and S genes of cyanobacteria are shuffled in E. coli and recombinants assayed as described in literature (Whitney S M and Andrews T J, op.cit). The selectivity of the shuffled enzyme for oxygenation vs. carboxylation is tabulated and quantitated.

Integrated Systems

The present invention provides computers, computer readable media and integrated systems comprising character strings corresponding to shuffled Calvin and krebs cycle enzymes such as Rubisco and corresponding enzyme-encoding nucleic acids. These sequences can be manipulated by in silico shuffling methods, or by standard sequence alignment or word processing software.

For example, different types of similarity and considerations of various stringency and character string length can be detected and recognized in the integrated systems herein. For example, many homology determination methods have been designed for comparative analysis of sequences of biopolymers, for spell-checking in word processing, and for data retrieval from various databases. With an understanding of double-helix pair-wise complement interactions among 4 principal nucleobases in natural polynucleotides, models that simulate annealing of complementary homologous polynucleotide strings can also be used as a foundation of sequence alignment or other operations typically performed on the character strings corresponding to the sequences herein (e.g., word-processing manipulations, construction of figures comprising sequence or subsequence character strings, output tables, etc.). An example of a software package with algorithms for calculating sequence similarity is BLAST, which can be adapted to the present invention by inputting character strings corresponding to the sequences herein.

BLAST is described in Altschul et al., J. Mol. Biol. 215:403-410 (1990). Software for performing BLAST analyses is publicly available through the National Center for Biotechnology Information (http://www.ncbi.nlm.nih.gov/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) of 10, a cutoff of 100, M=5, N=−4, and a comparison of both strands. For amino acid sequences, the BLASTP program uses as defaults a wordlength (W) of 3, an expectation (E) of 10, and the BLOSUM62 scoring matrix (see Henikoff & Henikoff (1989) Proc. Natl. Acad. Sci. USA 89:10915).

An additional example of a useful sequence alignment algorithm is PILEUP. PILEUP creates a multiple sequence alignment from a group of related sequences using progressive, pairwise alignments. It can also plot a tree showing the clustering relationships used to create the alignment. PILEUP uses a simplification of the progressive alignment method of Feng & Doolittle, J. Mol. Evol. 35:351-360 (1987). The method used is similar to the method described by Higgins & Sharp, CABIOS 5:151-153 (1989). The program can align, e.g., up to 300 sequences of a maximum length of 5,000 letters. The multiple alignment procedure begins with the pairwise alignment of the two most similar sequences, producing a cluster of two aligned sequences. This cluster can then be aligned to the next most related sequence or cluster of aligned sequences. Two clusters of sequences can be aligned by a simple extension of the pairwise alignment of two individual sequences. The final alignment is achieved by a series of progressive, pairwise alignments. The program can also be used to plot a dendogram or tree representation of clustering relationships. The program is run by designating specific sequences and their amino acid or nucleotide coordinates for regions of sequence comparison.

The shuffled enzymes of the invention, or corresponding coding nucleic acids, are optinally sequenced and the sequences aligned to provide structure-function information. For example, the alignment of shuffled sequences which are selected for conversion activity against the same target provides an indication of which residues are relevant for conversion of the target (i.e., conserved residues are likely more important for activity than non-conserved residues).

Standard desktop applications such as word processing software (e.g., Microsoft Word™ or Corel WordPerfect™) and database software (e.g., spreadsheet software such as Microsoft Excel™, Corel Quattro Pro™, or database programs such as Microsoft Access™ or Paradox™) can be adapted to the present invention by inputting character strings corresponding to shuffled Calvin or Krebs cycle enzymes such as Rubisco (or corresponding coding nucleic acids), e.g., shuffled by the methods herein. For example, the integrated systems can include the foregoing software having the appropriate character string information, e.g., used in conjunction with a user interface (e.g., a GUI in a standard operating system such as a Windows, Macintosh or LINUX system) to manipulate strings of characters. As noted, specialized alignment programs such as BLAST or PILEUP can also be incorporated into the systems of the invention for alignment of nucleic acids or proteins (or corresponding character strings).

Integrated systems for analysis in the present invention typically include a digital computer with software for aligning or manipulating sequences, as well as data sets entered into the software system comprising any of the sequences herein. The computer can be, e.g., a PC (Intel x86 or Pentium chip-compatible DOS™, OS2™ WINDOWS™ WINDOWS NT™, WINDOWS95™, WINDOWS98™ LINUX based machine, a MACINTOSH™, Power PC, or a UNIX based (e.g., SUN™ work station) machine) or other commercially common computer which is known to one of skill. Software for aligning or otherwise manipulating sequences is available, or can easily be constructed by one of skill using a standard programming language such as Visual basic, Fortran, Basic, Java, or the like.

Any controller or computer optionally includes a monitor which is often a cathode ray tube (“CRT”) display, a flat panel display (e.g., active matrix liquid crystal display, liquid crystal display), or others. Computer circuitry is often placed in a box which includes numerous integrated circuit chips, such as a microprocessor, memory, interface circuits, and others. The box also optionally includes a hard disk drive, a floppy disk drive, a high capacity removable drive such as a writeable CD-ROM, and other common peripheral elements. Inputting devices such as a keyboard or mouse optionally provide for input from a user and for user selection of sequences to be compared or otherwise manipulated in the relevant computer system.

The computer typically includes appropriate software for receiving user instructions, either in the form of user input into a set parameter fields, e.g., in a GUI, or in the form of preprogrammed instructions, e.g., preprogrammed for a variety of different specific operations. The software then converts these instructions to appropriate language for instructing the system to carry out any desired operation.

In one aspect, the computer system is used to perform “in silico” shuffling of character strings. A variety of such methods are set forth in “METHODS FOR MAKING CHARACTER STRINGS, POLYNUCLEOTIDES & POLYPEPTIDES HAVING DESIRED CHARACTERISTICS” by Selifonov and Stemmer, filed Feb. 5, 1999 (U.S. Ser. No. 60/118,854) and “METHODS FOR MAKING CHARACTER STRINGS, POLYNUCLEOTIDES & POLYPEPTIDES HAVING DESIRED CHARACTERISTICS” by Selifonov and Stemmer, filed Oct. 12, 1999 (U.S. Ser. No. 09/416,375). In brief, in the context of the present invention, genetic operators are used in genetic algorithms as described in the '375 application to change given ADPGPP sequences, e.g., by mimicking genetic events such as mutation, recombination, death and the like. Multi-dimensional analysis to optimize sequences can be also be performed in the computer system, e.g., as described in the '375 application.

A digital system can also instruct an oligonucleotide synthesizer to synthesize oligonucleotides, e.g., used for gene reconstruction or recombination, or to order oligonucleotides from commercial sources (e.g., by printing appropriate order forms or by linking to an order form on the internet).

The digital system can also include output elements for controlling nucleic acid synthesis (e.g., based upon a sequence or an alignment of a shuffled enzyme as herein), i.e., an integrated system of the invention optionally includes an oligonucleotide synthesizer or an oligonucleotide synthesis controller. The system can include other operations which occur downstream from an alignment or other operation performed using a character string corresponding to a sequence herein, e.g., as noted above with reference to assays.

Combination Shuffling

One aspect of the present invention, as noted, is the combinatorial shuffling of Rubisco and other enzymes which affect carbon fixation. For example, one aspect of the present invention involves separately or simultaneously shuffling Rubisco or any Calvin cycle enzyme or Krebs cycle enzyme in combination with Phosphoenolpyruvate (PEP) carboxylase (PEPC; EC 4.1.1.31). Considerable detail regarding PEPC gene shuffling is found in commonly assigned U.S. Patent Application U.S. Ser. No. 60/107,757 entitled “MODIFIED PHOSPHOENOLPYRUVATE CARBOXYLASE FOR IMPROVEMENT AND OPTIMIZATION OF PLANT PHENOTYPES” filed on 10 Nov. 1998 (Attorney Docket Number 018097-029100US) and in “MODIFIED PHOSPHOENOLPYRUVATE CARBOXYLASE FOR IMPROVEMENT AND OPTIMIZATION OF PLANT PHENOTYPES” co-filed on 9 Nov. 1999 (Attorney Docket Number 02-029100US) by Stemmer and Subramanian. Shuffled PEPC genes and shuffled Rubisco genes are optionally co-expressed in a cell or organism such as a plant to increase carbon fixation.

Similarly, shuffled Rubisco and shuffled ADP-glucose pyrophosphorylase (“ADPGPP”; EC 2.7.7.27; an enzyme involved in starch biosynthesis, e.g., in plants) can be expressed together in cells or plants to increase carbon fixation or to improve starch biosynthesis. Extensive details regarding ADP-glucose pyrophosphorylase gene shuffling are found in commonly assigned U.S. Patent Application U.S. Ser. No. 60/107,782, entitled “MODIFIED ADP-GLUCOSE PYROPHOSPHORYLASE FOR IMPROVEMENT AND OPTIMIZATION OF PLANT PHENOTYPES” filed on 10 Nov. 1998 (Attorney docket number 018097-029000US) and co-filed application “MODIFIED ADP-GLUCOSE PYROPHOSPHORYLASE FOR IMPROVEMENT AND OPTIMIZATION OF PLANT PHENOTYPES” filed on 10 Nov. 1999 (Attorney docket number 02-0290-1 US). Of course, shuffed Rubisco, ADPGPP, and PEPC can all be expressed together in a cell or organism such as a plant to increase carbon fixation, starch production, or the like.

In a further aspect, the present invention provides for the use of any apparatus, apparatus component, composition or kit herein, for the practice of any method or assay herein, and/or for the use of any apparatus or kit to practice any assay or method herein.

The foregoing description of the preferred embodiments of the present invention has been presented for purposes of illustration and description. They are not intended to be exhaustive or to limit the invention to the precise form disclosed, and many modifications and variations are possible in light of the above teaching.

Such modifications and variations which may be apparent to a person skilled in the art are intended to be within the scope of this invention.

All publications and patent applications herein are incorporated by reference to the same extent as if each individual publication or patent application was specifically and individually indicated to be incorporated by reference.

What is claimed is:

1. A method for obtaining an isolated polynucleotide encoding an enhanced Rubisco protein having Rubisco catalytic activity wherein the Km for CO₂ is significantly lower than a protein encoded by a parental polynucleotide encoding a naturally-occurring Rubisco enzyme, the method comprising:
recombining sequences of a plurality of parental polynucleotide species encoding at least one Rubsico sequence under conditions suitable for sequence shuffling to form a resultant library of sequence-shuffled Rubisco polynucleotides;
transferring said library into a plurality of host cells forming a library of transformants wherein sequence-shuffled Rubisco polynucleotides are expressed;
selecting for enhanced growth at low CO2/O2 ratios or assaying individual or pooled transformants for Rubisco catalytic activity to determine the relative or absolute Km for CO₂ and thereby identifying at least one enhanced transformant that expresses a Rubisco activity which has a significantly lower Km for CO₂ than the Rubisco activity encoded by the parental sequence(s);
recovering the sequence-shuffled Rubisco polynucleotide from at least one enhanced transformant.

2. The method of claim 1, further comprising the step of subjecting a recovered sequence-shuffled Rubisco polynucleotide encoding an enhanced Rubisco to at least one subsequent round of recursive shuffling and selection, wherein said recovered sequence-shuffled Rubisco polynucleotide is used as at least one parental sequence for subsequent shuffling.

3. The method of claim 1, wherein selection comprises assaying individual of pooled transformants for Rubisco catalytic activity to determine the relative or absolute Km for O₂ and identifying at least one enhanced transformant that expresses a Rubisco activity which has a significantly higher Km for O₂ than the Rubisco activity encoded by the parental sequence(s).

4. The method of claim 1, wherein selection comprises assaying individual or pooled transformants for Rubisco catalytic activity to determine the relative or absolute Km for O₂ and Km for CO₂ identifying at least one enhanced transformant that expresses a Rubisco activity which has a significantly lower ratio of Km for CO₂ to Km for O₂ than the Rubisco activity encoded by the parental sequence(s).

5. The method of claim 1, wherein selection comprises assaying samples of individual transformants and their clonal progeny which are isolated into discrete reaction vessels for Rubisco activity assay, or are assayed in situ.

6. The method of claim 1, wherein the host cell comprises a non-photosynthetic bacterium lacking an endogenous ribulose-5-phosphate kinase activity and is transformed with an expression cassette encoding the production of a functional ribulose-5-phosphate kinase (“R5PK”) activity, thereby forming an R5PK host cell, optionally including an expression cassette encoding a complementing Rubisco S subunit and, wherein selection comprises culturing the population of transformed R5P host cells in the presence of labelled carbon dioxide and/or labelled bicarbonate for a suitable incubation period, determining the amount of labelled carbon that is fixed by each transformed host cell and its clonal progeny relative to the amount of carbon fixed by untransformed R5PK host cells cultured under equivalent conditions.

7. The method of claim 6, wherein the R5PK host cells harbor expression cassettes encoding a complementing an L subunit and the library comprises shuffled S subunit encoding sequences.

8. The method of claim 6, wherein the host cell is a strain of non-photosynthetic bacterium which lacks endogenous phosphoglycerate kinase (PGK) activity and harbors an expression cassette encoding R5P kinase (R5PK) forming a PGK(−)/R5PK host cell.

9. The method of claim 8, wherein the host cell encodes a complementing subunit, and the method comprises the further step of culturing the population of transformed R5PK host cells in a minimal growth medium including glucose, wherein the minimal medium including glucose is insufficient to support the growth and replication of an untransformed PGK−/R5PK host cell, but is sufficient to support the growth and replication of a transformed PGK−/R5PK host cell expressing a functional Rubisco carboxylase activity.

10. A plant cell protoplast and clonal progeny thereof containing a sequence-shuffled polynucleotide encoding a Rubisco subunit which is not encoded by the naturally occurring genome of the plant cell protoplast.

11. A collection of plant cell protoplasts transformed with a library of sequence-shuffled Rubisco subunit polynucleotides in expressible form.

12. A regenerated plant containing at least one species of replicable or integrated polynucleotide comprising a sequence-shuffled portion and encoding a Rubisco subunit polypeptide.

13. A regenerated plant containing a polynucelotide expression cassette encoding a marine algal rbcL gene.

14. A regenerated plant of claim 13, further comprising a polynucleotide expression cassette encoding a marine algal rbcS gene.

15. A polynucleotide comprising: (1) a sequence encoding a shuffled Rubisco Form I L subunit gene (rbcL) linked to (2) a selectable marker gene which affords a means of selection when expressed in chloroplasts, and, optionally, flanked by (3) an upstream flanking recombinogenic sequence having sufficient sequence identity to a chloroplast genome sequence to mediate efficient recombination and (4) a downstream flanking recombinogenic sequence having sufficient sequence identity to a chloroplast genome sequence to mediate efficient recombination.

16. A polynucleotide of claim 15, wherein the polynucleotide encodes an enhanced Rubisco protein having Rubisco catalytic activity wherein the Km for CO₂ is significantly lower than a protein encoded by a parental polynucleotide encoding a naturally-occurring Rubisco enzyme.

17. A polynucleotide of claim 15, wherein the polynucleotide encodes an enhanced Rubisco protein having Rubisco catalytic activity wherein the Km for O₂ is significantly higher than a protein encoded by a parental polynucleotide encoding a naturally-occurring Rubisco enzyme or subunit.

18. A polynucleotide of claim 15, wherein the polynucleotide encodes an enhanced Rubisco protein having Rubisco catalytic activity wherein: (1) the Km for CO₂ is significantly lower than a protein encoded by a parental polynucleotide encoding a naturally-occurring Rubisco enzyme, (2) the Km for O₂ is significantly higher than a protein encoded by a parental polynucleotide encoding a naturally-occurring Rubisco enzyme, and/or (3) the ratio of the Km for CO₂ to the Km for O₂ is significantly lower than a protein encoded by a parental polynucleotide encoding a naturally-occurring Rubisco enzyme.

19. A method of producing a recombinant cell having an elevated carbon fixation activity, the method comprising:
(A) recombining one or more first Calvin or Krebs cycle enzyme coding nucleic acid, or a homologue thereof, with one or more first homologous nucleic acid to produce a library of recombinant first enzyme nucleic acid homologues;
(B) optionally repeating step (A) one or more times using one or more members of the library of recombinant first enzyme nucleic acid homologues as the one or more first enzyme coding nucleic acid which is active in the Calvin cycle, or the homologue thereof, or as the one or more first homologous nucleic acid, thereby producing a diversified library of recombinant first enzyme nucleic acid homologues;
(C) selecting the library of recombinant first enzyme nucleic acid homologues or the diversified library of recombinant first enzyme nucleic acid homologues for one or more of: an increased catalytic rate, an altered substrate specificity, and an increased ability of a cell expressing one or more members of the library to fix CO₂ when the one or more library members is expressed in the cell, thereby producing a selected library of recombinant first enzyme nucleic acid homologues; and,
(D) recursively repeating steps A-C one or more times, wherein the selected library of recombinant first enzyme nucleic acid homologues provides one or more of: the one or more first Calvin or Krebs cycle enzyme coding nucleic acid, the homologue thereof, or the one or more first homologous nucleic acid of step (A), wherein steps A-C are repeated until one or more members of the selected library produces an elevated carbon fixation level in a target recombinant cell when the one or more selected library member is expressed in the target cell, as compared to a carbon fixation activity of the target cell when the one or more selected library member is not expressed in the target cell.

20. The method of claim 1, wherein the one or more first Calvin or Krebs cycle enzyme, or the homologue thereof, or the one or more homologous first nucleic acid encodes a Rubisco enzyme, a Calvin cycle operon, or a homologue thereof.

21. The method of claim 19, wherein the recombining step is performed in vitro, in silico or in vivo, or a combination thereof.

22. The selected library of claim 19.

23. The one or more selected library member of claim 19.

24. The diversified library of claim 19.

25. The target recombinant cell of claim 19.

26. A plant comprising the target recombinant cell of claim 25.
